(19) United States
(12) Patent Application Publication
(10) Pub. No.: US 2024/0242834 A1
(43) Pub. Date: Jul. 18, 2024

(54) METHODS, SYSTEMS, AND APPARATUSES FOR PREVENTING DIABETIC EVENTS

(71) Applicants: Yixiang DENG, Washington, DC (US); George KARNIADAKIS, Providence, RI (US); Lu LU, Providence, RI (US); Christos MANTZOROS, Washington, DC (US); The United States of America as represented by the Department of Veterans Affairs, Washington, DC (US); BROWN UNIVERSITY, Providence, RI (US)

(72) Inventors: Yixiang Deng, Washington, DC (US); George Kamiadakis, Providence, RI (US); Lu Lu, Providence, RI (US); Christos Mantzoros, Providence, RI (US)

(21) Appl. No.: 18/289,499

(22) PCT Filed: May 3, 2022
(86) PCT No.: PCT/US2022/027397

§ 371 Date: Nov. 3, 2023

Related U.S. Application Data
(60) Provisional application No. 63/183,335, filed on May 3, 2021.

(51) Int. Cl. G16H 50/20; A61B 5/00; A61B 5/145; G16H 20/17

(52) U.S. Cl. CPC G16H 50/20; A61B 5/14532; A61B 5/7275; G16H 20/17

(57) ABSTRACT

Described herein are methods and systems for preventing a glycemic event. One or more improved deep-learning models for predicting glycemic events may receive current blood glucose data and other physiological data from associated with a patient. The one or more models may determine one or more future blood glucose values and whether or not the one or more future blood glucose values satisfy one ore more thresholds associated with one or more glycemic events (e.g., hypoglycemia and/or hyperglycemia). If the one or more future blood glucose values satisfy the one or more thresholds, one or more actions may be taken (e.g., administration of insulin).

### CROSS-REFERENCE TO RELATED APPLICATION

This application claims the priority benefit of U.S. Provisional Application No. 63/183,335, filed May 3, 2021, the entirety of which is incorporated herein by reference.

### BACKGROUND

Type 2 diabetes (T2D) is a multifactorial progressive chronic metabolic disorder, accounting for approximately 90% of all cases of diabetes. The prevalence of diabetes has been increasing rapidly over the past few decades. In 2019, about 463 million adults were living with diabetes, while it is estimated to be 578 and 700 million by 2030 and 2045, respectively. T2D and hyperglycemia are associated with an increased risk of vascular and non-vascular complications and premature mortality. Furthermore, evidence has also emphasized the importance of avoiding fluctuations in glycemia in T2D. Of note, the Advanced Technologies & Treatments for Diabetes (ATTD) consensus recommendations highlight the role of glycemic variability and the time in ranges (including the time in target range, hyperglycemia, and hypoglycemia) as key metrics for Continuous Glucose Monitoring (CGM). The available antidiabetic treatments combined with a near-to-normal glucose levels approach may lead to a lower frequency of T2D related microvascular and macrovascular events. On the other hand, intensified treatment targeting towards an intensive glucose control is associated with a higher risk of therapy-induced hypoglycemia and severe hypoglycemic events, which pose a potential risk for worsening or developing major macrovascular and microvascular complications, serious neurological consequences, as well as cardiovascular and all-cause mortality. Additionally, hypoglycemia is a severe adverse outcome that may negatively impact a patient's health and psychological status, leading to poor compliance and treatment adherence. Hypoglycemic events are also associated with a high direct and indirect cost for patients, healthcare systems, and society. Thus, the accurate prediction of blood glucose variations and, in particular, hypoglycemic events is of paramount importance to avoid potential detrimental complications and adjust the therapeutic strategy in a more optimized and personalized treatment strategy for patients with T2D. To this end, well developed predictive models with high sensitivity and accuracy, which are easy to implement, may facilitate better glycemic control, decrease the occurrence of hypoglycemic episodes or related complications and increase the quality of life in this population. Of note, due to the complexity of the blood glucose dynamics, the design of physiological models that produce an accurate prediction in every circumstance, e.g., hypo/normo/hyperglycemic events, is met with substantial restrictions.

However, this approach cannot capture the variability of blood glucose dynamics among different patients. Further, although in vitro experiments provide the most authentic environment to evaluate the performance of a control algorithm, it is also very expensive and risky. Hence, a number of in silico models have been proposed as a safe surrogates to evaluate the control algorithms of APs. However, these models either only simulate average population dynamics or consider patients in the ambulatory setting, where patients barely move, rather than those in a outpatient setting.

The prediction of blood glucose variations helps to adjust acute therapeutic measures and food intake in patients with type 2 diabetes. Therefore, predictive algorithms that are accurate and easy to implement may facilitate better glycemic control, decrease the occurrence of hypoglycemic episodes and increase the quality of life in this population.

These and other considerations are described herein.

### SUMMARY

It is to be understood that both the following general description and the following detailed description are exemplary and explanatory only and are not restrictive. The present methods and systems comprise one or more predictive models and/or one or more deep learning algorithms for patient-specific blood glucose level prediction. Specifically, the present methods and systems comprise a deep learning method to predict patient-specific blood glucose during various time horizons in the immediate future using patient-specific glucose measurements from the time period right before the prediction time period. Accurate prediction of blood glucose variations in type 2 diabetes (T2D) will facilitate better glycemic control and decrease the occurrence of hypoglycemic episodes as well as the morbidity and mortality associated with T2D, hence increasing the quality of life of patients. Due to the complexity of the blood glucose dynamics, it is difficult to design accurate predictive models in every circumstance, e.g., hypo/normo/hyperglycemic events. Further, deep learning models usually require a large amount of data to train the networks, and therefore, they are usually trained by population level data rather than individual level data. The present methods and systems result, in one example, from the inventors realization that the major challenges to address in blood glucose dynamics are (1) datasets are often too small to train a patient-specific predictive model, and (2) datasets are usually highly imbalanced given that hypo- and hyperglycemic episodes are usually much less common that normoglycemia. Described herein is a system and methodology comprising transfer learning and data augmentation. The systems and methods described herein may be implemented to address the fundamental problems of small and imbalanced datasets in many other biomedical applications for predicting the outcomes of diseases, e.g., prevention of acute complications such as hypoglycemia or diabetic ketoacidosis in type 1 diabetes (T1D) patients by achieving a flexible, fast and effective control of blood glucose levels.

Described herein are methods and systems for improved deep-learning models. In one example, a plurality of data records and a plurality of variables may be used by a computing device to generate and train a deep-learning model, such as a predictive model. The computing device may determine a numeric representation for each data record of a first subset of the plurality of data records. Each data record of the first subset of the plurality of data records may comprise a label, such as a binary label (e.g., yes/no, hypo/non-hypo), a multi-class label (hypo/normo/hyper) and/or a percentage value. The computing device may determine a numeric representation for each variable of a first subset of the plurality of variables. Each variable of the first subset of the plurality of variables may comprise the label (e.g., the binary label and/or the percentage value).

The computing device may determine a plurality of features for the predictive model. The computing device may train the predictive model. The computing device may output the predictive model following the training. The predictive model—once trained—may be capable of providing a range of predictive data analysis.

The computing device may use a trained predictive model to determine one or more of a prediction or a score associated with the first data record. Trained predictive models as described herein may be capable of providing a range of predictive and/or generative data analysis. The trained predictive models may have been initially trained to provide a first set of predictive and/or generative data analysis, and each may be retrained in order to provide another set of predictive and/or generative data analysis. Once retrained, predictive models described herein may provide another set of predictive and/or generative data analysis. Retraining may refer to using a transfer learning method. Additional advantages of the disclosed methods and systems will be set forth in part in the description which follows, and in part will be understood from the description, or may be learned by practice of the disclosed method and systems.

### BRIEF DESCRIPTION OF THE DRAWINGS

The accompanying drawings, which are incorporated in and constitute a part of the present description serve to explain the principles of the methods and systems described herein:

FIG. 1 shows an example method;

FIG. 2 shows an example method;

FIG. 3 shows an example method;

FIG. 4 shows an example method;

FIG. 5 shows an example system;

FIG. 6 shows an example system;

FIG. 7A shows an example data profile;

FIG. 7B shows an example method;

FIG. 8A shows an example network architecture model;

FIG. 8B shows an example unit;

FIG. 8C shows an example network architecture model;

FIG. 9A shows an example network architecture model;

FIG. 9B shows an example network architecture model;

FIG. 9C shows an example network architecture model;

FIG. 10A shows an example unit;

FIG. 10B shows an example unit;

FIG. 11 shows an example method;

FIG. 12 shows an example method;

FIG. 13 shows an example data profile;

FIG. 14 shows an example chart;

FIG. 15 shows example charts;

FIGS. 16A-16D show example tables;

FIG. 17 shows an example chart;

FIG. 18 shows example curves;

FIG. 19 shows an example chart and graph;

FIG. 20 shows example charts;

FIG. 21 shows example tables;

FIG. 22 shows show example charts;

FIG. 23 shows an example table;

FIG. 24 shows example tables;

FIG. 25 shows example graphs;

FIG. 26 shows an example graphs;

FIG. 27 shows an example table;

FIG. 28 shows an example table;

FIG. 29 shows an example table;

FIG. 30 shows example charts;

FIG. 31 shows example charts;

FIG. 32 shows an example table;

FIG. 33 shows an example table;

FIG. 34 shows an example table;

FIG. 35 shows an example table;

FIG. 36 shows example charts;

FIG. 37 shows example charts;

FIG. 38 shows example charts;

FIG. 39 shows example curves;

FIG. 40 shows example curves;

FIG. 41 shows an example table;

FIG. 42 shows an example model;

FIG. 43 shows show example equations;

FIG. 44 shows example methods;

FIG. 45 shows an example algorithm;

FIG. 46 shows an example table;

FIG. 47 shows example equations;

FIG. 48 shows an example table;

FIG. 49 shows show example charts;

FIG. 50 shows example charts;

FIG. 51 shows example charts;

FIG. 52 shows example charts;

FIG. 53 shows example charts;

FIG. 54 shows example equations;

FIG. 55 shows an example model;

FIG. 56 shows example charts;

FIGS. 57A-57B show example equations; and

FIG. 58 shows predictive thresholds.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium may be implemented. Any suitable computer-readable storage medium may be utilized including hard disks, CD-ROMs, optical storage devices, magnetic storage devices, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

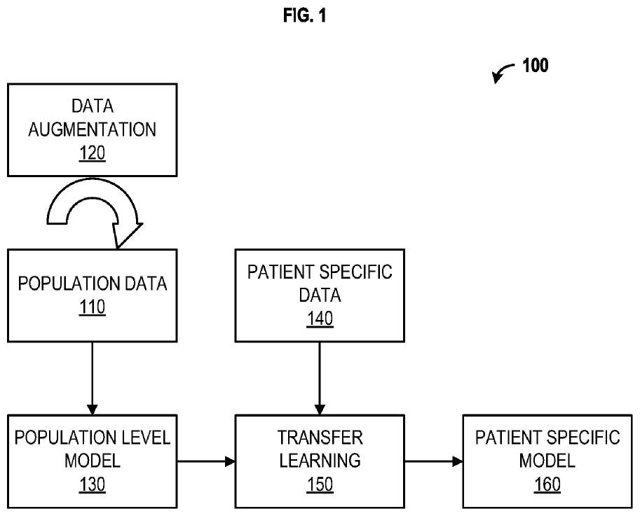

FIG. 1 shows an example method 100. The method 100 may comprise predicting and preventing one or more glycemic events. The method 100 may be carried (e.g., executed on) any one or more devices of the present disclosure or any combination thereof. The method may be carried out by system of devices. For example, the system may comprise a glucose sensor, a control algorithm, and an insulin infusion device. The system may be referred to as an artificial pancreas. The artificial pancreas (AP) is a closed-loop system designed for patients with T1DM to improve their BG regulation, and consequently decrease the risk of diabetic complications. The method 100 may result in appropriate insulin treatment comprising dosing adjustments of insulin (e.g., exogenous insulin administered by a user and/or insulin administered by a device such as an insulin pump) to account for changes in one or more physiological parameters such as carbohydrates intake, physical exercise, and illness or stress.

The method 100 may comprise training one or more predictive models. The one or more predictive models may be trained so as to determine a likelihood of a glycemic event or other medical event. The glycemic event may comprise a glycemic episode wherein a blood glucose value associated with a patient falls below or rises above one or more blood glucose thresholds. For example, the glycemic event may comprise a hypoglycemic event wherein the blood glucose value associated with the patient falls below 70 mg/dL. For example, the glycemic event may comprise a hyperglycemic event wherein the blood glucose value associated with the patient rises above 120 mg/dL. The aforementioned ranges are merely exemplary and explanatory. The one or more blood glucose thresholds may be any value. Further, the method may comprise adjusting, on a per-patient basis, the one or more blood glucose thresholds. For example, the artificial pancreas may adjust the one or more thresholds based on patient-specific data gathered, for example, from a history of physiological parameter inputs. The artificial pancreas may comprise one or more offline reinforcement learning algorithms (e.g., batch constrained Q learning, or “BCQ”) where the parameters are inferred with system biology informed neural networks (SBINNs) and patient specific data. The method may be executed on a variety of hardware and software components as described herein including various network structures and learning methods.

At 110, population data may be determined. The population data may include medical data associated with a plurality of persons. For example, the population data may include one or more blood glucose histories of the plurality of persons. The one or more blood glucose histories may comprise, for each person of the plurality of persons, blood glucose levels and associated information such as temporal information (e.g., times and dates at which the one or more blood glucose levels for each person of the plurality of persons is determined), weight, carbohydrate intake, exercise, stress, levels interstitial blood glucose measures, plasma glucose measures, subcutaneous cell glucose measures, combinations thereof, or the like. Additional medical history data may also be included in the population data, such as for each person of the plurality of persons, demographic data such as age and gender, body composition data such as body mass, height, BMI and similar information, hormonal levels including cortisol, leptin, fasting glucose, insulin, HOMA 1-IR, and blood glucose data brief data such as data reading length (hours), model input blood glucose (BG) length (minutes), hypoglycemia threshold (mg/dL), hyperglycemia threshold (mg/dL), and HbA1c (%). Determining the population data may comprise receiving the population data from a database. For example, a database operated by a healthcare facility or network may be queried and in response to the query, the population data may be received. The population data may comprise a publicly available dataset including continuous glucose monitoring, insulin, physiological sensor, and self-reported life-event data

Optionally, at 120, the population data may be augmented. Augmenting the population data may comprise performing, on the population data, one or more data augmentation methods so as to augment minority class data. Minority class data may comprise data (e.g., samples) associated with hypoglycemic events and hyperglycemic events. Conversely, majority class data may comprise data (e.g., samples) associated with normoglycemic events (e.g., blood glucose levels in the population data associated with the plurality of patients which are determined to fall within a normal range). The one or more data augmentation methods may comprise, for example, oversampling by repeating, Gaussian noise, using Time-series Generative Adversarial Networks (TimeGAN), and/or mixup. A policy may be determined to select one or more data augmentation methods to be implemented. The policy may include different hyperparameters to determine a number of augmentation methods as well as a type of composition. The selection of the policy can be made while the model is being trained.

At 130, a population level model may be determined. The population level model may comprise training one or more neural networks on the population data in order to determine (e.g., predict) the one or more glycemic events. Training the population level model may, for example, comprise a leave-one-out method wherein the one or more networks is initially trained on the population data after removing data associated with a target patient.

At 140, patient specific data may be determined. The patient specific data may comprise data associated with the target patient (e.g., the one patient “left out” out of the population data at step 130, or a patient currently being monitored by a CGM device). Data associated with the target patient may be split into two datasets: a training dataset and a testing dataset. For example, if, for a given target patient, there exists 1500 data samples, (e.g., 1500 blood glucose measurements and associated data), the training dataset may comprise 1000 data samples of the 1500 data samples and the testing dataset may comprise the remaining 500 data samples. The training dataset may comprise data samples temporally prior (e.g., earlier in time) to the testing dataset.

Optionally, at 150, a transfer learning method may be executed. The transfer learning method may comprise retraining the one or more neural networks on the training dataset of the target patient data and then testing the retrained one or more neural networks on the testing dataset of the target patient data. The transfer learning method may comprise any of the transfer learning methods described further herein including Transfer1, Transfer2, Transfer3, and/or Pre-training. Retraining the one or more neural networks may comprise reusing neural network parameters (e.g., the weights and biases) inherited from the population level training. Retraining the one or more neural networks may comprise re-initializing the network parameters. Retraining the one or more neural networks may comprise either maintaining (e.g., “fixing”) or tuning weights and biases associated with one or more edges in the one or more neural networks trained on the population data. Transfer learning may comprise transferring to a target task, knowledge associated with a source task (e.g., source task knowledge). For example, the source task may comprise determining relationships between glycemic events in the population data and other associated data (e.g., the hormonal data or other data). For example, the target task may comprise predicting glycemic events based on current patient data. For example, the source task knowledge may comprise the network parameters (e.g., the weights and biases) associated with the model trained on population data. The source task knowledge may be replicated, replicated in part, or tuned in order to apply the source task knowledge to the target task.

Transfer learning in neural networks refers to an adaptation for using a result obtained by learning transfer source data items (e.g., the population level data), in feature extraction such as classification (e.g., hypo/hyper or non) or regression of transfer target data items. For example, a transfer learning method may incorporate a multi-layer neural network that has been trained through deep learning by using transfer source data items wherein the multi-layer neural network is further trained in order to be adaptive to transferred target data items (e.g., the target patient BG data). Specifically, a first multi-layer neural network, which is a multi-layer neural network trained by using a plurality of first data items, is prepared. In transfer learning, the configuration of some of the layers of the first multi-layer neural network is changed to obtain a new multi-layer neural network. The new multi-layer neural network is trained by using a plurality of second data items to obtain a second multi-layer neural network. The plurality of first data items serve as transfer source data items, whereas the plurality of second data items serve as transfer target data items. In transfer learning, lower layers from the input layer to a certain hidden layer of the multi-layer neural network that has been trained through deep learning are used as a general-purpose feature extractor without modifying the configuration thereof. In contrast, upper layers from a hidden layer that accepts an output of the certain hidden layer to the output layer of the multi-layer neural network are replaced with newly configured adaptive layers (that is, new hidden and output layers), and the adaptive layers are trained by using the transfer target data items. For example, the first multi-layer neural network that includes a plurality of layers (e.g., C1 to C5, (wherein C denotes a convolutional layer) and FC6 to FC8 (wherein FC denotes a fully connected layer)) and that has been trained by using the plurality of first data items, which serve as transfer source data items (a large number of available labeled images), is prepared. The FC block (e.g., FC6 to FC8) may be replaced. Additionally and/or alternatively, the layer FC8 may be removed from the first multi-layer neural network, and two adaptive layers FCa and FCb are added to obtain a new multi-layer neural network. The new multi-layer neural network is then trained by using the plurality of second data items, which serve as transfer target data items, to obtain the second multi-layer neural network. The aforementioned is merely exemplary and explanatory and a person skilled in the art will appreciate that any suitable network architecture and learning methodology may be implemented in order to enable the present disclosure.

At step 160, a patient specific model may be determined. The patient specific model may be determined based on the transfer learning (e.g., retraining) as described above. The patient specific model may be configured to receive current blood glucose (BG) data associated with a current patient (e.g., the target patient). The current BG data may comprise, for example one or more blood glucose measurements associated with the current patient. The current BG data may comprise one or more physiological parameters as well. For example, the current BG data may comprise carbohydrate intake, stress, exercise, weight, interstitial blood glucose measures, plasma glucose measures, subcutaneous cell glucose measures, combinations thereof, or the like. The one or more blood glucose measurements may comprise any number of measurements determined during a period of time. For example, the current BG data may comprise recently determined BG measurements. For example, the recently determined BG measurements may comprise BG measurements determined every 5 minutes for the previous 35 minutes (e.g., the most recent 7 BG measurements associated with the current patient). The patient specific model may be configured to predict, based on the current BG data, whether a glycemic event will occur.

The patient-specific model may receive physiological data associated with one or more physiological parameters. For example, the one or more physiological parameters may comprise exogenous insulin administration, food intake (e.g., carbohydrate intake), and exercise.

Insulin may be administered based on the patient specific model including the one or more physiological parameters. For example, the patient specific module may receive the one or more current blood glucose values and the one or more physiological parameters and determine one or more future blood glucose levels. For example, the model may predict, based on the one or more current blood glucose levels and the one or more physiological parameters a blood glucose value at some point in the future (e.g., one or more future blood glucose values).

The one or more future blood glucose values may satisfy one or more thresholds. For example, the threshold may be blood glucose levels below 70 mg/dl and/or above 120 mg/dl. The aforementioned thresholds are merely exemplary and explanatory. The one or more thresholds may be adjusted (e.g., by the predictive model, by a care provider, by a patient) on a patient-specific basis. If one or more of the one or more future blood glucose values satisfies the one or more thresholds (e.g., if a future blood glucose value is low) the insulin may be caused to be administered. For example, an insulin administration signal may be sent to an insulin pump, wherein the insulin administration signal is configured to cause the insulin pump to administer insulin. The amount of insulin administered may be determined based on the one or more future blood glucose values. The amount of insulin administered may be determined so as to avoid one or more glycemic events such as hyperglycemia or hypoglycemia on a patient-specific basis.

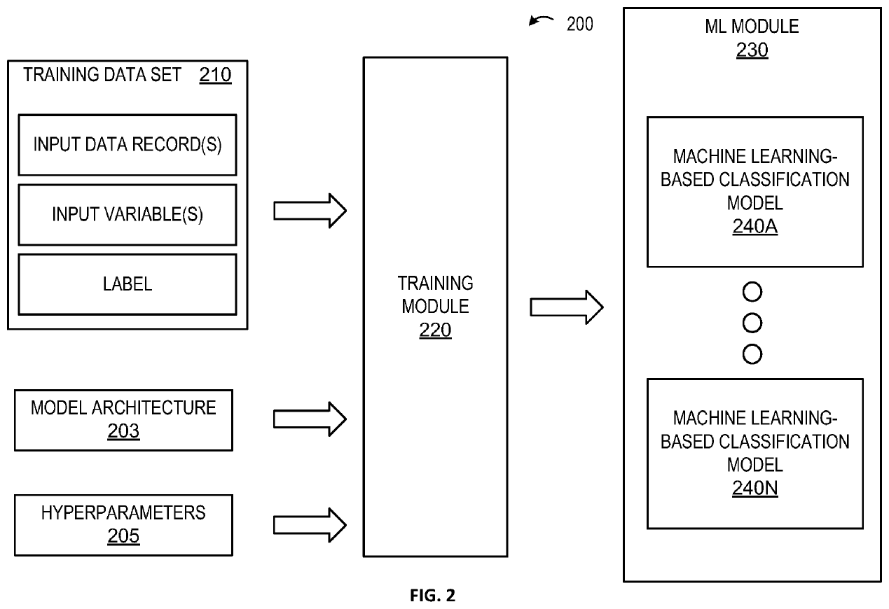

Turning now to FIG. 2, an example method 200 is shown. The method 200 may be performed based on an analysis of one or more training data sets 210 by a training module 220, at least one ML module 230 that is configured to provide one or more of a prediction or a score associated with data records and one or more corresponding variables. The training module 220 may be configured to train and configure the ML module 230 using one or more hyperparameters 205 and a model architecture 203. The model architecture 203 may comprise a predictive model as described herein. The hyperparameters 205 may comprise a number of neural network layers/blocks, a number of neural network filters (e.g., convolutional filters) in a neural network layer, a number of epochs etc. Each set of the hyperparameters 205 may be used to build the model architecture 203, and an element of each set of the hyperparameters 205 may comprise a number of inputs (e.g., data record attributes/variables) to include in the model architecture 203. For example, the first set of hyperparameters may be associated with a first model. The first model may be associated with a first task (e.g., a source task). The first task may comprise population level analysis. The second set of hyperparameters may be associated with a second model. The second model may be associated with a second task (e.g., the target task). The second task may comprise patient level analysis. For example, in the case of predicting diabetic events, an element of a first set of the hyperparameters 205 may comprise data associated with data records for a population (e.g., the population data comprise both the majority class data and the minority class data). An element of a second set of the hyperparameters 205 may comprise a data record for a particular patient and/or all demographic attributes (e.g., variable attributes) associated with that particular patient. In other words, an element of each set of the hyperparameters 205 may indicate that as few as one or as many as all corresponding attributes of the data records and variables are to be used to build the model architecture 203 that is used to train the ML module 230.

The training data set 210 may comprise one or more input data records associated with one or more labels (e.g., a binary label (yes/no, hypo/non-hypo), a multi-class label (e.g., hypo/non/hyper) and/or a percentage value). The label for a given record and/or a given variable may be indicative of a likelihood that the label applies to the given record. A subset of the data records may be randomly assigned to the training data set 210 or to a testing data set. In some implementations, the assignment of data to a training data set or a testing data set may not be completely random. In this case, one or more criteria may be used during the assignment. In general, any suitable method may be used to assign the data to the training or testing data sets, while ensuring that the distributions of yes and no labels are somewhat similar in the training data set and the testing data set.

The training module 220 may train the ML module 230 by extracting a feature set from a plurality of data records (e.g., labeled as yes, hypo/hyper, no for normo) in the training data set 210 according to one or more feature selection techniques. The training module 220 may train the ML module 230 by extracting a feature set from the training data set 210 that includes statistically significant features of positive examples (e.g., labeled as being yes) and statistically significant features of negative examples (e.g., labeled as being no).

The training module 220 may extract a feature set from the training data set 210 in a variety of ways. The training module 220 may perform feature extraction multiple times, each time using a different feature-extraction technique. In an example, the feature sets generated using the different techniques may each be used to generate different machine learning-based classification models 240A-240N. For example, the feature set with the highest quality metrics may be selected for use in training. The training module 220 may use the feature set(s) to build one or more machine learning-based classification models 240A-240N that are configured to indicate whether a particular label applies to a new/unseen data record based on its corresponding one or more variables.

The training data set 210 may be analyzed to determine any dependencies, associations, and/or correlations between features and the yes/no labels in the training data set 210. The identified correlations may have the form of a list of features that are associated with different yes/no labels. The term “feature,” as used herein, may refer to any characteristic of an item of data that may be used to determine whether the item of data falls within one or more specific categories. A feature selection technique may comprise one or more feature selection rules. The one or more feature selection rules may comprise a feature occurrence rule. The feature occurrence rule may comprise determining which features in the training data set 210 occur over a threshold number of times and identifying those features that satisfy the threshold as candidate features.

Two commonly-used retraining approaches are based on initialization and feature extraction. In the initialization approach the whole network is further trained, while in the feature extraction approach the last few fully-connected layers are trained from a random initialization, and other layers remain unchanged. In addition to these two approaches, a third approach may be implemented by combining these two approaches (e.g., the last few fully-connected layers are further trained, and other layers remain unchanged).

A single feature selection rule may be applied to select features or multiple feature selection rules may be applied to select features. The feature selection rules may be applied in a cascading fashion, with the feature selection rules being applied in a specific order and applied to the results of the previous rule. For example, the feature occurrence rule may be applied to the training data set 210 to generate a first list of features. A final list of candidate features may be analyzed according to additional feature selection techniques to determine one or more candidate feature groups (e.g., groups of features that may be used to predict whether a label applies or does not apply). Any suitable computational technique may be used to identify the candidate feature groups using any feature selection technique such as filter, wrapper, and/or embedded methods. One or more candidate feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., yes/no).

As another example, one or more candidate feature groups may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. As an example, forward feature selection may be used to identify one or more candidate feature groups. Forward feature selection is an iterative method that begins with no feature in the machine learning model. In each iteration, the feature which best improves the model is added until an addition of a new variable does not improve the performance of the machine learning model. As an example, backward elimination may be used to identify one or more candidate feature groups. Backward elimination is an iterative method that begins with all features in the machine learning model. In each iteration, the least significant feature is removed until no improvement is observed on removal of features. Recursive feature elimination may be used to identify one or more candidate feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients. For example, the regression model may comprise the following preconditions: the prediction horizon is 20 minutes if not mentioned otherwise and the hypoglycemia threshold is set at 80 mg/dL.

After the training module 220 has generated a feature set(s), the training module 220 may generate one or more machine learning-based classification models 240A-240N based on the feature set(s). A machine learning-based classification model may refer to a complex mathematical model for data classification that is generated using machine-learning techniques. In one example, the machine learning-based classification model 240 may include a map of support vectors that represent boundary features. By way of example, boundary features may be selected from, and/or represent the highest-ranked features in, a feature set.

The training module 220 may use the feature sets extracted from the training data set 210 to build the one or more machine learning-based classification models 240A-240N for each classification category (e.g., yes, no, hypo/non, hypo/non/hyper). In some examples, the machine learning-based classification models 240A-240N may be combined into a single machine learning-based classification model 240. Similarly, the ML module 230 may represent a single classifier containing a single or a plurality of machine learning-based classification models 240 and/or multiple classifiers containing a single or a plurality of machine learning-based classification models 240.

The extracted features (e.g., one or more candidate features) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting ML module 230 may comprise a decision rule or a mapping for each candidate feature.

In an embodiment, the training module 220 may train the machine learning-based classification models 240 as a convolutional neural network (CNN). The CNN may comprise at least one convolutional feature layer and three fully connected layers leading to a final classification layer (softmax). The final classification layer may finally be applied to combine the outputs of the fully connected layers using softmax functions as is known in the art. A grid search method may be implemented to obtain the optimal hyperparameters of the CNN. Grid search is a tuning technique configured to compute the optimum values of hyperparameters. Grid search is an exhaustive search performed on a specific hyperparameter's value of a model. The CNN may include a gated linear unit layer. The CNN may receive an input (e.g., an order 1 or higher tensor). The input then sequentially goes through a series of processing. One processing step is usually called a layer, which could be a convolution layer, a pooling layer, a normalization layer, a fully connected layer, a loss layer, etc. The Rectified Linear Unit (hence the name ReLU) can be regarded as a truncation performed individually for every element in the input. For example, the BG sequence may be first fed into the first CNN layer of the model and a first convolutional layer is applied to the sequence. Next, a gated linear unit may be applied to the output of the first convolutional layer. These two steps can be repeated, and the final output from the last CNN layer is fed into the fully-connected layers.

The candidate feature(s) and the ML module 230 may be used to predict whether a label applies to a data record in the testing data set. In one example, the result for each data record in the testing data set includes a confidence level that corresponds to a likelihood or a probability that the one or more corresponding variables are indicative of the label applying to the data record in the testing data set. The confidence level may be a value between zero and one, and it may represent a likelihood that the data record in the testing data set belongs to a yes/no status with regard to the one or more corresponding variables (e.g., diabetic event). In one example, when there are two statuses (e.g., yes and no), the confidence level may correspond to a value p, which refers to a likelihood that a particular data record in the testing data set belongs to the first status (e.g., yes). In this case, the value 1−p may refer to a likelihood that the particular data record in the testing data set belongs to the second status (e.g., no). In general, multiple confidence levels may be provided for each data record in the testing data set and for each candidate feature when there are more than two labels. A top performing candidate feature may be determined by comparing the result obtained for each test data record with the known yes/no label for each data record. In general, the top performing candidate feature will have results that closely match the known yes/no labels. The top performing candidate feature(s) may be used to predict the yes/no label of a data record with regard to one or more corresponding variables. For example, a new data record may be determined/received. The new data record may be provided to the ML module 230 which may, based on the top performing candidate feature, classify the label as either applying to the new data record or as not applying to the new data record.

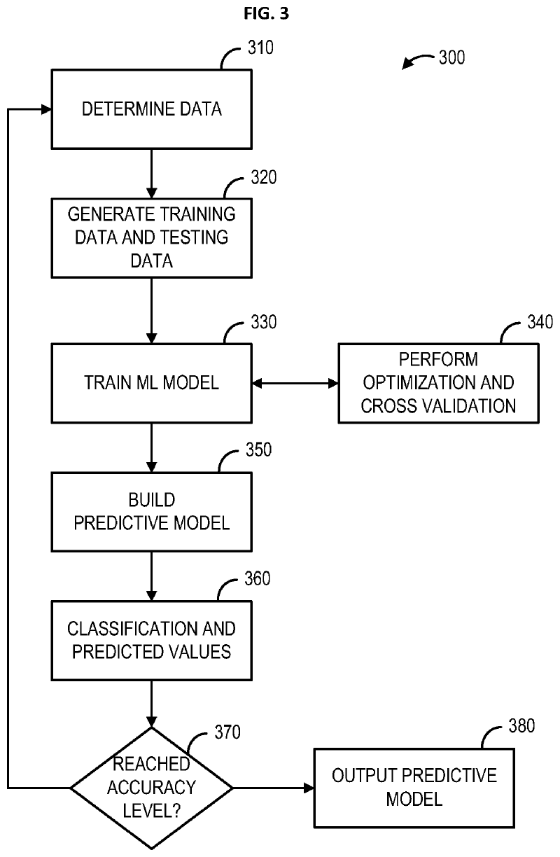

Turning now to FIG. 3, a flowchart illustrating an example training method 300 for generating the ML module 230 using the training module 620 is shown. The training module 220 can implement supervised, unsupervised, and/or semi-supervised (e.g., reinforcement based) machine learning-based classification models 240A-240N. The training module 220 may comprise a data processing module and/or a predictive module. The method 300 illustrated in FIG. 3 is an example of a supervised learning method; variations of this example of training method are discussed below, however, other training methods can be analogously implemented to train unsupervised and/or semi-supervised machine learning models.

The training method 300 may determine (e.g., access, receive, retrieve, etc.) first data records that have been processed by the data processing module at step 310. The first data records may comprise a labeled set of data records. The labels may correspond to a label (e.g., yes or no). The training method 300 may generate, at step 320, a training data set and a testing data set. The training data set and the testing data set may be generated by randomly assigning labeled data records to either the training data set or the testing data set. In some implementations, the assignment of labeled data records as training or testing samples may not be completely random. As an example, a majority of the labeled data records may be used to generate the training data set. For example, 65% of the labeled data records may be used to generate the training data set and 35% may be used to generate the testing data set. The training data set may comprise population data that excludes data associated with a target patient.

The training method 300 may train one or more machine learning models at step 330. In one example, the machine learning models may be trained using supervised learning. In another example, other machine learning techniques may be employed, including unsupervised learning and semi-supervised. The machine learning models trained at 330 may be selected based on different criteria depending on the problem to be solved and/or data available in the training data set. For example, machine learning classifiers can suffer from different degrees of bias. Accordingly, more than one machine learning model can be trained at 330, optimized, improved, and cross-validated at step 340.

For example, a loss function may be used when training the machine learning models at step 330. The loss function may take true labels and predicted outputs as its inputs, and the loss function may produce a single number output. The present methods and systems may implement a mean absolute error, relative mean absolute error, mean squared error and relative mean squared error using the original training dataset without data augmentation. In particular, the performance of models with different loss functions using four classification metrics, e.g., sensitivity, positive predictive value (PPV), specificity and negative predictive value (NPV). Results of experimentation indicate that the model using relative mean absolute error (REL. MAE) outperforms models using the other three loss functions, because the model using the relative mean absolute error maintains a balanced high value for each of the aforementioned four metrics. Given the regression setup, the REL. MAE is implemented in the loss function and the real-valued prediction is then categorized into “hypoglycemia” or “no hypoglycemia” by the hypoglycemia threshold (80 mg/dL). In some embodiments employing data augmentation methods (e.g., data augmentation 120 of FIG. 1), four classification metrics are computed, e.g., sensitivity, the recall of the positive class; PPV, positive predictive value denoting the precision of the positive class; specificity, the recall of the negative class; NPV, negative predictive value denoting the precision of the negative class. The positive class denotes the hypoglycemia class, and the negative class denotes the “no hypoglycemia” class. The minority data fold represents the number of copies of hypoglycemia samples in the training data after data augmentation. “Raw” denotes no data augmentation on the training dataset, e.g., the training data is intact; 2-fold means the raw minority data is kept in the training data and another copy of minority data is generated by either repeating or synthesizing in each training epoch. For all four methods, as more minority samples (hypoglycemia samples) are introduced in the training data by data augmentation methods such as repeating or TimeGAN or mixup, the positive predictive value and the specificity decrease while the sensitivity increases, which is similar to the effect of increasing the Gaussian noise level on a copy of the minority data. The model was run with each augmentation method for 5 times to obtain the mean and standard deviation of the classification metrics.

One or more minimization techniques may be applied to some or all learnable parameters of the machine learning model (e.g., one or more learnable neural network parameters) in order to minimize the loss. For example, the one or more minimization techniques may not be applied to one or more learnable parameters, such as encoder modules that have been trained, a neural network block(s), a neural network layer(s), etc. This process may be continuously applied until some stopping condition is met, such as a certain number of repeats of the full training dataset and/or a level of loss for a left-out validation set has ceased to decrease for some number of iterations. In addition to adjusting these learnable parameters, one or more of the hyperparameters 205 that define the model architecture 203 of the machine learning models may be selected. The one or more hyperparameters 205 may comprise a number of neural network layers, a number of neural network filters in a neural network layer, etc. For example, as discussed above, each set of the hyperparameters 205 may be used to build the model architecture 203, and an element of each set of the hyperparameters 205 may comprise a number of inputs (e.g., data record attributes/variables) to include in the model architecture 203. The element of each set of the hyperparameters 205 comprising the number of inputs may be considered the “plurality of features” as described herein. That is, the cross-validation and optimization performed at step 340 may be considered as a feature selection step. An element of a second set of the hyperparameters 305 may comprise data record attributes for a particular patient. In order to select the best hyperparameters 205, at step 340 the machine learning models may be optimized by training the same using some portion of the training data (e.g., based on the element of each set of the hyperparameters 205 comprising the number of inputs for the model architecture 203). The optimization may be stopped based on a left-out validation portion of the training data. A remainder of the training data may be used to cross-validate. This process may be repeated a certain number of times, and the machine learning models may be evaluated for a particular level of performance each time and for each set of hyperparameters 205 that are selected (e.g., based on the number of inputs and the particular inputs chosen).

A best set of the hyperparameters 205 may be selected by choosing one or more of the hyperparameters 205 having a best mean evaluation of the “splits” of the training data. This function may be called for each new data split, and each new set of hyperparameters 205. A cross-validation routine may determine a type of data that is within the input (e.g., attribute type(s)), and a chosen amount of data (e.g., a number of attributes) may be split-off to use as a validation dataset. A type of data splitting may be chosen to partition the data a chosen number of times. For each data partition, a set of the hyperparameters 205 may be used, and a new machine learning model comprising a new model architecture 203 based on the set of the hyperparameters 205 may be initialized and trained. After each training iteration, the machine learning model may be evaluated on the test portion of the data for that particular split. The evaluation may return a single number, which may depend on the machine learning model's output and the true output label. The evaluation for each split and hyperparameter set may be stored in a table, which may be used to select the optimal set of the hyperparameters 205. The optimal set of the hyperparameters 205 may comprise one or more of the hyperparameters 205 having a highest average evaluation score across all splits.

The training method 300 may select one or more machine learning models to build a predictive model at 350. The predictive model may be evaluated using the testing data set. The predictive model may analyze the testing data set and generate one or more of a prediction or a score at step 360. The one or more predictions and/or scores may be evaluated at step 370 to determine whether they have achieved a desired accuracy level. Performance of the predictive model may be evaluated in a number of ways based on a number of true positives, false positives, true negatives, and/or false negatives classifications of the plurality of data points indicated by the predictive model.

For example, the false positives of the predictive model may refer to a number of times the predictive model incorrectly classified a label as applying to a given data record when in reality the label did not apply. Conversely, the false negatives of the predictive model may refer to a number of times the machine learning model indicated a label as not applying when, in fact, the label did apply. True negatives and true positives may refer to a number of times the predictive model correctly classified one or more labels as applying or not applying. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives. When such a desired accuracy level is reached, the training phase ends and the predictive model (e.g., the ML module 230) may be output at step 380; when the desired accuracy level is not reached, however, then a subsequent iteration of the training method 300 may be performed starting at step 310 with variations such as, for example, considering a larger collection of data records.

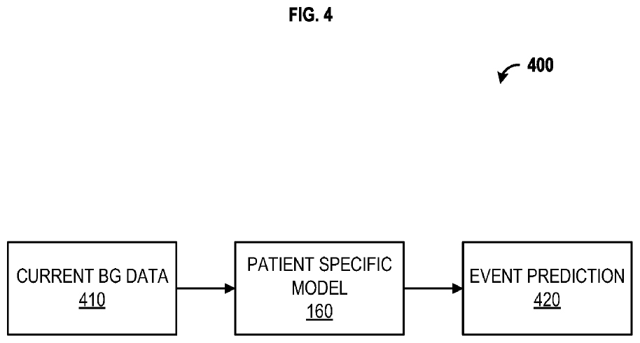

FIG. 4 shows an example method 400. The method 400 may facilitate the prediction of a glycemic event. The method 400 may be carried out on (e.g., facilitated by) any one or more devices described herein. For example, the method 400 may be carried out on a single computing device or a combinations of devices. The method 400 may be carried by a system referred to as an artificial pancreas. For example, the system may comprise a glucose sensor, a control algorithm, and an insulin infusion device. The artificial pancreas (AP) is a closed-loop system designed for patients with T1DM to improve their BG regulation, and consequently decrease the risk of diabetic complications.

At step 410, current BG data can be determined. The current BG data may comprise, for example one or more blood glucose measurements associated with a current patient. The one or more blood glucose measurements may comprise any number of measurements determined during a period of time. For example, the current BG data may comprise recently determined BG measurements. For example, the recently determined BG measurements may comprise BG measurements determined every 5 minutes for the previous 35 minutes (e.g., the most recent 7 BG measurements associated with the current patient). The patient specific model 160 may be configured to predict, based on the current patient BG data, whether a glycemic event will occur for that patient. The current BG data may comprise carbohydrate intake, stress, exercise, weight, interstitial blood glucose measures, plasma glucose measures, subcutaneous cell glucose measures, combinations thereof, or the like.

The current BG data can be inputted into the patient specific model 160 and at step 420, an event prediction can be made. The event prediction may comprise a prediction as to whether or not a glycemic event (e.g., hyperglycemic episode or hypoglycemic episode) is likely to occur. The glycemic event may comprise a glycemic episode wherein a blood glucose value associated with a patient falls below or rises above one or more thresholds. For example, the glycemic event may comprise a hypoglycemic event wherein a predicted blood glucose value associated with the patient falls below 80 mg/dL. For example, the glycemic event may comprise a hyperglycemic event wherein a predicted blood glucose value associated with the patient rises above 180 mg/dL. The method 400 may be executed on a variety of hardware and software components as described herein including various network structures and learning methods.

The method may further comprise causing, based on the event prediction an administration, or cancellation of administration, of insulin. For example, if the event prediction comprises a hypoglycemic event, the method may comprise causing an administration of insulin via an insulin pump in an amount sufficient to prevent the hypoglycemic event. Additionally or alternatively, causing the administration of insulin may comprise outputting a prompt configured to suggest to a user to administer insulin in an amount sufficient to prevent the hypoglycemic event. For example, if the event prediction comprises a hyperglycemic event, the method comprise determining a scheduled administration of insulin, and cancelling the scheduled administration of insulin.

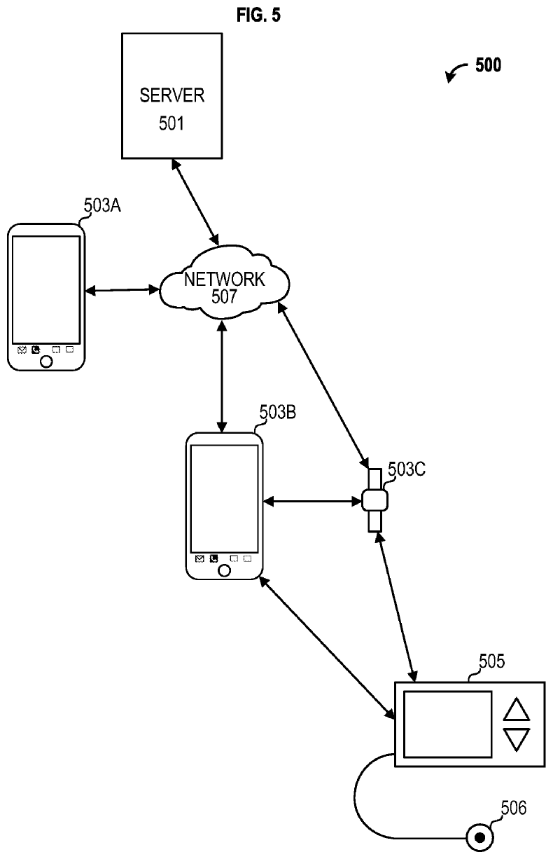

FIG. 5 shows an example system 500. The system 500 may comprise a server 501, one or more user devices 503A, 503B, and 503C (the user device 503C may comprise a wearable device) and a pump/monitor device 505. The pump/monitor device 505 may comprise a single device or one or more devices (e.g., a pump device and/or a monitor device). The server 501, the one or more user devices 503A-C, and the pump/monitor device 505 may be configured to communicate via a network 507. The server 501 may comprise one or more computing devices (e.g., one or more servers). The server 501 may be configured to store and/or execute the one or more neural networks and/or the one or more models (e.g., the population level model 130 and/or the patient specific model 160). The user devices 503A and/or 503B may comprise one or more computing devices (e.g., a smartphone, a tablet, and the like). The user device 503C may comprise a device configured to determine a current BG value and to communicate the current BG value to another device. The user device 503C may be, for example, a smart watch, a smart ring, a smart pendant, and the like. The user device 503C may be configured with a sensor to determine the current BG value. The sensor may be, for example, a blood glucose meter such as a constant glucose meter (CGM). The blood glucose meter be configured to pass a signal through a sample of fluid (e.g., blood). The signal may comprise an electrical current. The blood glucose meter may be configured to determine, based on a measured property of the signal, the current BG value. For example, the pump/monitor device 505 may comprise a sensor/pump 506 under the skin of the current patient. The sensor/pump 506 may be configured to measure interstitial glucose levels and determine the current BG value. The pump/monitor device 505 may be part of an insulin pump or a separate device, which the current patient might carry in a pocket or purse. The pump/monitor device 505 may be configured to send information directly to a smartphone or tablet (e.g., the user devices 503A and 503B).

The pump/monitor device 505 may be configured to administer insulin. The pump/monitor device 505 may be configured to administer insulin based on a current BG value and/or based on a predicted future BG value. The pump/monitor device 505 may be configured to administer basal insulin and/or bolus insulin. For example, the pump/monitor device 505 may be configured to administer the basal insulin and/or the bolus insulin in accordance with a standard, for example, a standard promulgated by the American Diabetes Association. For example, the pump/monitor device 505 may be configured to receive a current BG reading indicating the current BG value and/or dietary information and determine, based on the current BG reading and/or dietary information, an amount of insulin to be administered, and administer the amount of insulin. The amount of insulin to be administered may be determined according to standard algorithms per the American Diabetes Association Guidelines. For example, the pump/monitor device 505 may be configured to deliver 1 unit per hour (“U/h”) from 9 am-5 pm and 0.7 U/h from 5 pm-9 am. For example, a user may configure the pump/monitor device 505 with a temporary basal rate (“temp basal”) for specific activities, like exercise. For example, the user might program a 50% reduction in basal rate for a long bike ride. Additionally and/or alternatively a user device such as any of the user devices 503A-C can determine a wearer's activity and accordingly determine an adjustment in insulin administration. After a set period of time, or based on a determination that increased insulin is no longer required, the pump/monitor device 505 may return to the normal pattern. With regards to bolus insulin, the pump/monitor device 505 may be configured to adjust one or more bolus insulin settings. The one or more bolus insulin settings may comprise target blood glucose range, insulin-to-carbohydrate ratio (I:C), insulin sensitivity factor (ISF) or correction factor, duration of insulin action (DIA), and/or insulin on board. The target blood glucose range may comprise a desired blood glucose level. For example, to correct a high blood sugar level, one unit of insulin may administered to drop blood glucose by 50 mg/dl. The target blood glucose range may be entered into the pump/monitor device 505's settings as a single target for an entire day or one or more targets corresponding to one or more time periods. A bolus calculator may use the target blood glucose range to determine how much correction insulin to recommend in cases of high blood sugar. For example, if the target blood glucose level is set at 100 mg/dl, and the current BG value is 175 mg/dl, a bolus calculator will recommend more correction bolus insulin to reduce blood glucose by 75 mg/dl. The bolus calculator may be configured to receive an ISF measurement. The ISF may represent how much one unit of insulin is expected to lower blood sugar. For example, if 1 unit of insulin will drop the patient's blood sugar by 25 mg/dl, then the patient's insulin sensitivity factor is 1:25. In the example above, the pump would recommend 3 units of insulin to bring blood glucose from 175 mg/dl down to 100 mg/dl. The aforementioned are merely exemplary and explanatory and a person skilled in the art will appreciate that any rate of administration during any time period may be implemented.

In operation, one or more of the user device 503C and/or the pump/monitor device 505 may determine data (e.g., a current blood glucose measurement) associated with, for example, a person wearing the wearable device 503C and/or the pump/monitor device 505. For example, the wearable device 503C may be configured to determine blood glucose measurements by a non-invasive technique and/or configured for Constant Glucose Monitoring (CGM). For example, via an optical/infrared sensor and/or a sensor configured to transmit low-power radio waves

The user devices 503A-B may be configured to receive current blood glucose measurements from one or more of the wearable device 503C and/or the pump/monitor device 505. The user devices 503A-C may comprise a user interface. The user interface may be configured to display current blood glucose measurements and any other data or information. For example, the user interface may comprise a graphical user interface which may display the current blood glucose measurements. Any or all of the user device 503A-C and the pump/monitor device 505 may be configured to send current blood glucose measurements to the server 501 via the network 507.

The user devices 503A-C may be configured to receive dietary data and/or exercise data. For example, the user device 503A and 503B may comprise an image module and an image recognition module. The image module may be configured to capture, or otherwise determine (e.g., receive) one or more images. For example, the image module may comprise a camera configured to take pictures. For example, the image module may comprise a mobile application configured to display images of food or other information associated with food (e.g., names of dishes, caloric content, carbohydrate content, etc.). The images of food may be selectable. For instance, if a user is about to eat cookies, the user may take a picture of the cookies or select an image of cookies from the mobile application. In the scenario where the user captures an image of the cookies, the image recognition module may receive the image, perform image recognition analysis (as is known in the art), and determine that the image contains an image of cookies. Based on the determination, the user device 503A or the user device 503B may query a database (either locally on the user device or remotely, for example, on the server 501) to determine (e.g., retrieve) the additional information associated with the cookies. The additional information may be used to determine, based on the patient specific predictive model, how consuming the cookies may impact the user's blood glucose levels in the near future.

The user devices 503A-C may be configured with a scanning module. The scanning module may be configured to scan a code (e.g., a barcode, QR code, or other similar code) associated with the food (e.g., found on packaging of the food). Based on scanning the code, a user device of the user devices 503A-C may query a database containing the additional information associated with the food. In response to the query, the user device may receive the additional information and determine, based on the patient specific model, how consuming the food will impact the user's blood glucose levels in the near future. The user device may be configured to receive one or more messages (e.g., from another device of the system 500). For example, upon determining the future blood glucose event (e.g., value) satisfies one or more thresholds, the serve 501 may send a message to the user device 503, wherein the message is configured to cause the user device to output a prompt, alarm, message, combinations thereof, and the like. The prompt may include the current BG data and future BG data.

The server 501 may be configured to receive the current BG data and execute the method 400 in order to predict an event. The server 501 may be configured to send information to any or all of the user devices 503A-C and the pump/monitor device 505. For example, after determining an event prediction, the server 501 may send a message to any or all of the user devices 503A-C and/or the pump/monitor device 505. For example, the message may comprise an alert which may indicate to the current patient or some other person (e.g., a caretaker) that a glycemic event is imminent. In another example, the message may comprise an instruction sent to the pump/monitor device 505. The instruction may comprise an instruction to administer an amount of insulin. The system 500 may also comprise a smart pen. The smart pen may be configured to receive a message. For example, any of the server 501 or the user devices 503A-C may send the message to the smart pen. The message may comprise a command. The command may cause the smart pen to administer a dose of insulin based on the prediction of the glycemic event.

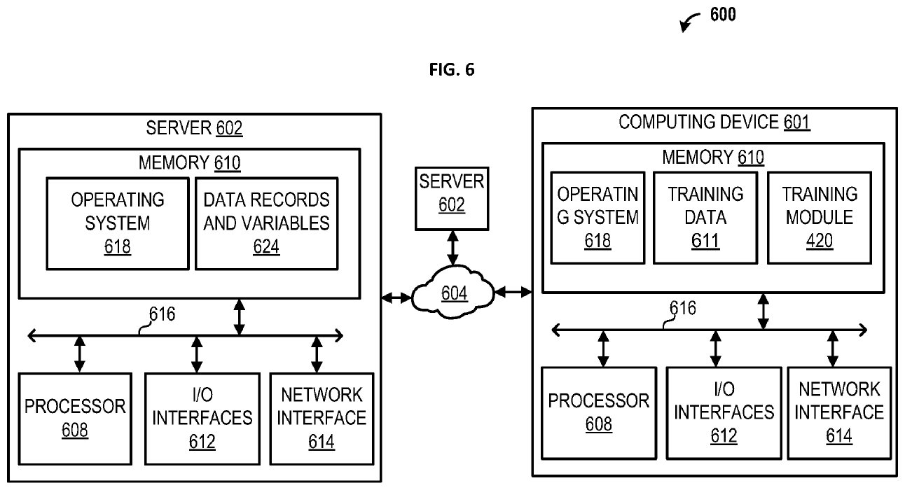

FIG. 6 is a block diagram depicting an environment 600 comprising non-limiting examples of a computing device 601 (e.g., a remote computing device such as a medical database associated with a healthcare provider or academic institute or any of the user devices 503A-C and/or the pump/monitor device 505 of FIG. 5) and a server 602 (e.g., the server 501 of FIG. 5) connected through a network 604. In an aspect, some or all steps of any described method herein may be performed by the computing device 601 and/or the server 602. The computing device 601 can comprise one or multiple computers configured to store one or more of the data records, training data 210 (e.g., labeled data records), and various modules to execute the methods described herein. The server 602 can comprise one or multiple computers configured to store the data records. Multiple servers 602 can communicate with the computing device 601 via the through the network 604. In an embodiment, the computing device 601 may comprise a repository for training data 611 generated by the methods described herein.

The computing device 601 and the server 602 can be a digital computer that, in terms of hardware architecture, generally includes a processor 608, memory system 610, input/output (I/O) interfaces 612, and network interfaces 614. These components (608, 610, 612, and 614) are communicatively coupled via a local interface 616. The local interface 616 can be, for example, but not limited to, one or more buses or other wired or wireless connections, as is known in the art. The local interface 616 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 608 can be a hardware device for executing software, particularly that stored in memory system 610. The processor 608 can be any custom made or commercially available processor, a central processing unit (CPU), an auxiliary processor among several processors associated with the computing device 601 and the server 602, a semiconductor-based microprocessor (in the form of a microchip or chip set), or generally any device for executing software instructions. When the computing device 601 and/or the server 602 is in operation, the processor 608 can be configured to execute software stored within the memory system 610, to communicate data to and from the memory system 610, and to generally control operations of the computing device 601 and the server 602 pursuant to the software.

The I/O interfaces 612 can be used to receive user input from, and/or for providing system output to, one or more devices or components. User input can be provided via, for example, a keyboard and/or a mouse. System output can be provided via a display device and a printer (not shown). I/O interfaces 612 can include, for example, a serial port, a parallel port, a Small Computer System Interface (SCSI), an infrared (IR) interface, a radio frequency (RF) interface, and/or a universal serial bus (USB) interface.

The network interface 614 can be used to transmit and receive from the computing device 601 and/or the server 602 on the network 604. The network interface 614 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 614 may include address, control, and/or data connections to enable appropriate communications on the network 604.

The memory system 610 can include any one or combination of volatile memory elements (e.g., random access memory (RAM, such as DRAM, SRAM, SDRAM, etc.) and nonvolatile memory elements (e.g., ROM, hard drive, tape, CDROM, DVDROM, etc.). Moreover, the memory system 610 may incorporate electronic, magnetic, optical, and/or other types of storage media. Note that the memory system 610 can have a distributed architecture, where various components are situated remote from one another, but can be accessed by the processor 608.

The software in memory system 610 may include one or more software programs, each of which comprises an ordered listing of executable instructions for implementing logical functions. In the example of FIG. 6, the software in the memory 610 of the computing device 601 can comprise the training data 611, a training module 620, and a suitable operating system (O/S) 618. In the example of FIG. 6, the software in the memory system 810 of the server 802 can comprise data records and variables 624 (e.g., the data records 104 and the variables 105), and a suitable operating system (O/S) 618. The operating system 618 essentially controls the execution of other computer programs and provides scheduling, input-output control, file and data management, memory management, and communication control and related services.

For purposes of illustration, application programs and other executable program components such as the operating system 618 are illustrated herein as discrete blocks, although it is recognized that such programs and components can reside at various times in different storage components of the computing device 601 and/or the server 602. An implementation of the training module 220 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

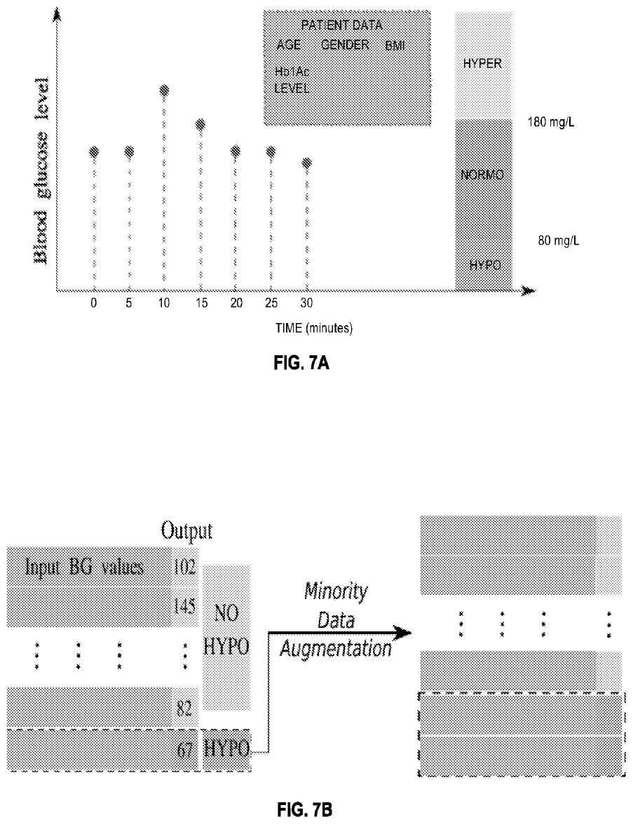

FIG. 7A shows an example data profile. The data profile may comprise information associated with patient's blood glucose levels over time (every 5-min measurements) during several time periods in the past (e.g., 30 mins) along with key and widely available patient's personal data to predict the patient's blood glucose level in the future (e.g., 30 min later). In particular, the objective of the present methods and systems is to detect hyperglycemia (HYPER, blood glucose level>180 mg/dL) and hypoglycemia (HYPO, blood glucose level<80 mg/dL), and normal (NORMO, normoglycemia, 80 mg/dL≤blood glucose level≤180 mg/dL). The present methods and systems address the following two classification tasks of diabetic blood glucose, e.g., one classification is “hypoglycemia” vs. “no hypoglycemia” and the other is “hypoglycemia” vs. “normoglycemia” vs. “hyperglycemia.” The threshold for hyperglycemia may be set to 180 mg/dL, e.g., blood glucose levels higher than 180 mg/dL may be labeled with “hyperglycemia”. On the other hand, the threshold for hypoglycemia may be 80 mg/dL. That is so say, the system may label blood glucose levels lower than 80 mg/dL as “hypoglycemia”. Here, unlike the common definition for level 1 hypoglycemia based on the threshold of 70 mg/dL, 80 mg/dL may be chosen as the hypoglycemia threshold. The outcome of interest in is the prediction of future BG values, e.g., 5, 10, 15, 20, 25, 30, 45, 60 min later. For example, one or more sample measure of BG measured within 30 minutes (after 7 BG values) may comprise one input data segment and predict the future BG level. Further additional predictor patients' characteristics like BMI, age, gender and Hb1Ac may be incorporated by prefixing the scaled characteristic values in the beginning of their BG sequence.

FIG. 7B shows an example pre-processing flow involving minority data augmentation. In addition to the problem of small data, another challenge in diabetic blood glucose prediction is the data imbalance. In particular, the dataset of normal-level blood glucose measurements (called majority class) is orders-of-magnitude larger than the dataset of blood glucose measurements with specific symptom (called minority class), e.g., hypoglycemia. The model trained on the imbalanced dataset leads to a biased performance, e.g., the accuracy of the minority class is much worse than that of the majority class. To address the data imbalance issue, various general approaches have been developed, including pre-processing approaches, algorithmic centered approaches, and hybrid approaches, but learning from imbalanced data effectively and efficiently is still an open problem. The methods and systems described herein may comprise one or pre-processing approaches to address a data imbalance issue, because they are only performed on training data and can be directly combined with any neural network algorithm. Methods may include re-sampling, Gaussian noise, mixup, and time-series generative adversarial networks.

The preprocessing flow may comprise a minority data augmentation step. Given the need to detect hypoglycemia more accurately and robustly, data augmentation on the minority class, e.g., augment the hypoglycemia samples in the training dataset, is an effective way of enforcing the neural network to learn the underlying patterns of the hypoglycemia data at a finer scale compared to learning on the dataset without data augmentation. Implementing data augmentation on the minority class (hypoglycemic labels) using synthetic data (not oversampling by repeating) increases the model sensitivity in detecting hypoglycemia, from less than 80% to more than 96% depending on the specific augmentation method for a prediction horizon of 20 minutes. Thus, the present methods and systems, by performing data augmentation on the minority class, may facilitate early treatment intervention and prevention of potential hypoglycemic events and hence is a significant improvement preferred in clinical diagnosis given the fatal consequences of hypoglycemia for patients with serious complications caused by type 2 diabetes.

The minority data augmentation step may comprise one or more data augmentation methods. For example, the one or more data augmentation methods may comprise one or more of oversampling by repeating, Gaussian noise, mixup, or TimeGAN.

Oversampling by repeating may comprise repeating minority class samples (e.g., the input-out pairs where the output BG data indicates less than 80 mg/dL or greater than 180 mg/dL) in the training data set (e.g., the population level data) for k folds (e.g., for 2-fold oversample by repeating, the minority samples are duplicated once such that the minority data is doubled in the augmented population level data to be used for training. Hence, for k-fold oversampling by repeating, the minority class of the population data may be augmented by adding k−1 copies of the minority class data (e.g., data labeled as either hypoglycemic or hyperglycemic) to the population data.

The one or more data augmentation methods may comprise adding (e.g., “infusing”) Gaussian white noise to the training dataset. One or more levels of Gaussian noise may be used. For example, noise with variance at 5, 10, and 50 mg/dL may be infused to the input BG data of the minority class, whose output BG value may be below the hypoglycemia threshold (e.g., there are two copies of minority training in the augmented dataset, one is the original copy collected by devices like CGMs or retrieved from one or more databases, and the other is a copy generated by infusing Gaussian white noises). Similarly, white noise may be added with variance at 190, 200, 220 mg/dL or any other level above the hyperglycemia threshold.

The present systems and methods comprise a mixup methodology which may linearly interpolate between samples in the training dataset. For example, the methods may linearly interpolate between samples in the training dataset (e.g., the minority class) using the following formula:

$$\tilde{x} = λ {x}_{i} + ( 1 - λ ) {x}_{j} ,$$

$$\tilde{y} = λ {y}_{i} + ( 1 - λ ) {y}_{j} ,$$

where x^{˜}, y^{˜} denote generated input and output, respectively; λ is a hyperparameter following the Beta distribution, Beta(α, α); xi, xⱼ denote inputs from two different samples and yi, yj denote the corresponding output of those two different samples.

The data augmentation method may comprise k-fold mixup. By k-fold mixup, the size of the minority class is increased to k times of its original size by adding k−1 copies of synthetic data using mixup for each training epoch. The original mixup algorithm does not include k as a hyperparameter, e.g., in the original mixup, the original training data is replaced by synthetic data generated by linear interpolation in the beginning of each training epoch.

The hyper-parameter α in the Beta distribution Beta(α, α) of mixup may be a sensitive parameter controlling the diversity of the synthetic samples, e.g., higher α produces samples more resembling to the reference real data while lower α introduces samples very different from the reference real data. With α=1, Beta(1, 1) is equivalent to a uniform random distribution. The mixup method may comprise one or more hyperparameters.

The present methods and systems may implement data transforms (e.g., data augmentations) which preserve the labels of the data and improve estimation by enlarging the span of the training data. For example, the present methods and systems preserve the labels of the data by only augmenting the minority training data, which consequently increases the span of minority data, by generating synthetic data using Gaussian noise, TimeGAN or mixup. Further, implementing synthetic minority data (data generated by infusing Gaussian noise, TimeGAN or mixup) may increase the span of minority data much more significantly than repeating the original minority data.

Data augmentation may further comprise using a time-series generative adversarial networks (TimeGAN). For example, one or more synthetic minority samples may be generated using TimeGAN on the data of minority class and compared the performance of models when different folds of synthetic data of minority class were added to augmented dataset in the beginning of each training epoch. The TimeGAN model may generate blood glucose sequences with hypoglycemia labels very similar to the true blood glucose sequences fed into the model. For example, in addition to the unsupervised adversarial loss on both real and synthetic sequences, a stepwise supervised loss may be introduced using the original data as supervision, thereby explicitly encouraging the model to capture stepwise conditional distributions in the data. This takes advantage of the fact that there is more information in the training data than simply whether each datum is real or synthetic. Thus, a model may learn from the transition dynamics from real sequences. The TimeGAN data augmentation methodology may comprise an embedding network to provide a reversible mapping between features and latent representations, thereby reducing the high-dimensionality of the adversarial learning space. This capitalizes on the fact the temporal dynamics of even complex systems are often driven by fewer and lower-dimensional factors of variation. Importantly, the supervised loss is minimized by jointly training both the embedding and generator networks, such that the latent space not only serves to promote parameter efficiency—it is specifically conditioned to facilitate the generator in learning temporal relationships. This framework may be generalized to handle the mixed-data setting, where both static and time-series data can be generated at the same time.

TimeGAN may comprise an embedding function, recovery function, sequence generator, and sequence discriminator. The embedding and recovery functions provide mappings between feature and latent space, allowing the adversarial network to learn the underlying temporal dynamics of the data via lower-dimensional representations. The discriminator may comprise a network characterized as a function which maps from source data to a probability that the source data is from the real data distribution. That is to say, the TimeGAN network may capture one or more statistical distributions associated with the training data and use those statistical distributions to synthesize samples from a learned distribution.

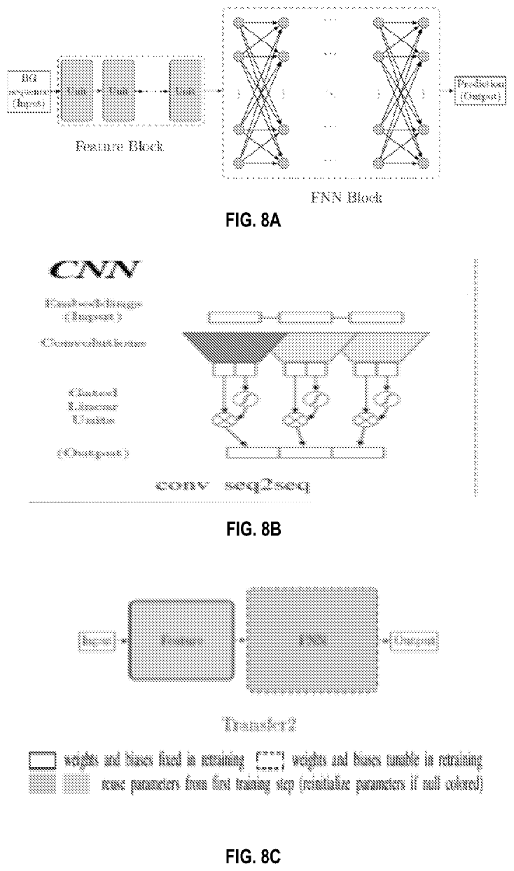

FIG. 8A shows an example network architecture. The network architecture may comprise a neural network. The neural network may comprise a feature block and a fully connected feed-forward neural network. The unit in the feature block may be model specific. For example, in the convolutional neural network, the unit may comprise a convolutional sequence to sequence (conv seq2seq) unit.

FIG. 8B shows an example network architecture unit (e.g., conv seq2seq). The network architecture may comprise a convolutional neural network (CNN). The network may receive one or more inputs. For example, the one or more inputs may comprise embeddings. The one or more inputs may comprise population data. The population data may comprise a minority class. The minority class may comprise data related to hypoglycemic events. The population data may comprise an augmented minority class. For example, the minority class data may be augmented according to mixup and TimeGAN methodologies as described herein to create synthetic minority samples. In the gated CNN, convolution kernels may be implemented which create hierarchical representations over an input time series, in which nearby BG measurements interact at lower layers while distant BG measurements interact at higher layers. For example, network architecture may comprise a CNN with kernel size may be 4, with four conv seq2seq units; a FNN width of 10, and 3 FNN layers. The transfer learning methodology may comprise reuse of the weights of the feature block and FNN block, used to retrain the FNN block. In gated CNNs, convolutional kernels create hierarchical representations over the input time series, in which nearby BG measurements interact at lower layers while distant BG measurements interact at higher layers. In CNNs, the input sequence may be fed into the network simultaneously, and thus an embedding of the position of input elements may be implemented.

The methods and systems described herein may comprise one or more transfer learning techniques. FIG. 8C shows a transfer learning method called Transfer2. Transfer2 may comprise a feature block wherein weights and biases are fixed in retraining and wherein parameters are reused from a first training step. Transfer2 may comprise an FNN wherein weights and biases are tunable in retraining and wherein parameters are reused from the first training step. For example, as shown in FIG. 8C, colored blocks represent reusing neural network parameters (weights and biases) inherited from the pre-training step, and blocks bounded with solid lines represent those blocks, of which the network parameters are frozen during retraining step. To address the problem of small dataset, transfer learning can be employed, which stores knowledge gained while solving one problem (e.g., population data) and then applying it to a different but related problem (e.g., patient-specific data). Transfer learning may comprise two-step training. In step 1, the models may be trained with the data from all the patients except for this target patient. In step 2, the models obtained from step 1 may be re-trained with the training data from this target patient. In transfer learning, the leave-one-out method may be implemented, while the training procedure of neural networks includes two steps: first, the neural network may be pretrained, (e.g., take one patient out of the group as the target patient and pretrain the neural networks on the rest of the unchosen patients). The target patient's data may be split into two parts, the training dataset and the testing dataset. The network may be retrained on the training dataset of the target patient. This may be repeated for other patients in the group one-by-one and obtain the prediction accuracy for each of the patients in the group on their own testing dataset. In the aforementioned pretraining step, the effect was tested by augmenting the cohort data with an open dataset, which monitors the BG level in children and adolescents with type 1 diabetes. Two commonly-used retraining approaches are based on initialization and feature extraction. In the initialization approach the whole network is further trained, while in the feature extraction approach the last few fully-connected layers are trained from a random initialization, and other layers remain unchanged. Additionally, a third method may be implemented by combining these two approaches, e.g., the last few fully-connected layers are further trained, and other layers remain unchanged. Other transfer learning methods may include pretrain, Transfer1, Transfer3, combinations thereof, and the like.

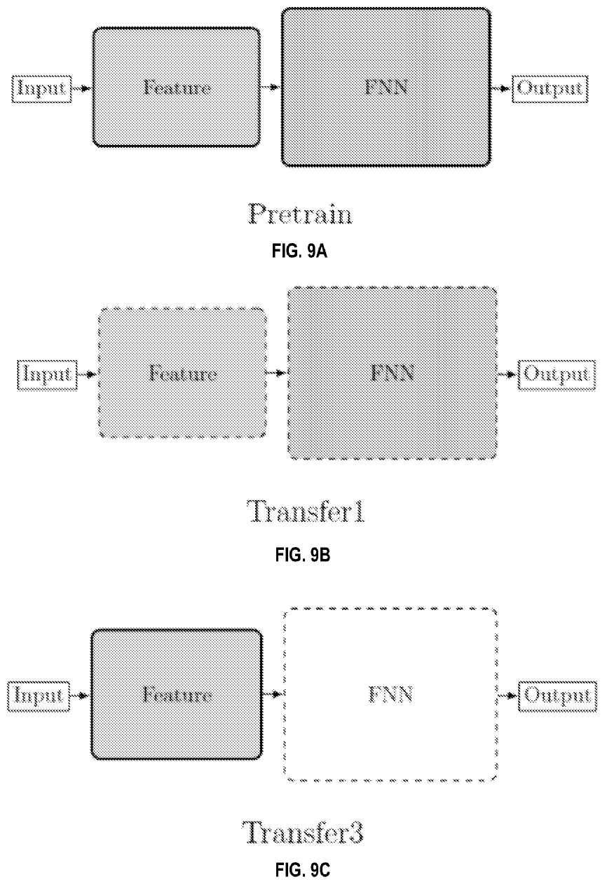

FIG. 9A shows an example pretrain learning method. Pretrain may comprise a feature block wherein weights and biases are fixed in retraining. The feature block may reuse parameters from a first training step. Pretrain may comprise an FNN wherein weights and biases are fixed in retraining and wherein the FNN may reuse parameters from the first training step.

FIG. 9B shows an example Transfer1 learning method. Transfer1 may comprise a feature block wherein weights and biases are tunable in retraining and wherein parameters are reused from the first training step. Transfer1 may comprise an FNN wherein weights and biases are tunable in retraining and wherein parameters may be reused from the first training step.

FIG. 9C shows an example Transfer3 learning method called. Transfer3 may comprise a feature block wherein weights and biases are fixed in training and wherein parameters may be reused from the first training step. Transfer 3 may comprise an FNN wherein weights and biases are tunable in retraining and wherein parameters may be reinitialized from the first training step.

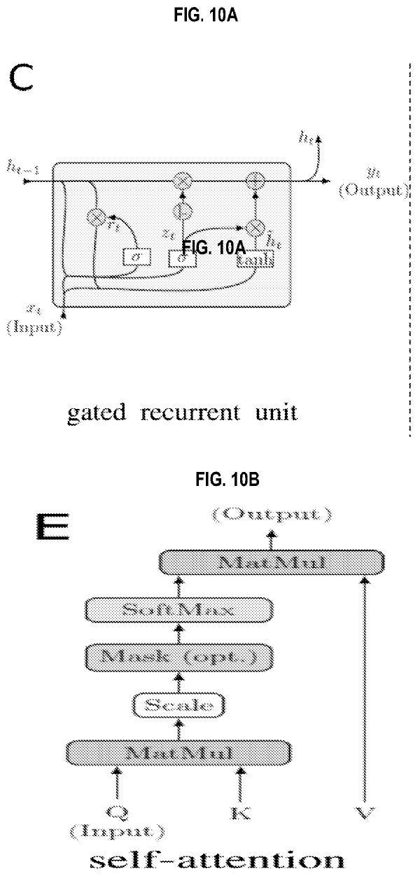

FIG. 10A shows an example gate recurrent unit. The gated recurrent unit may be used in a recurrent neural network (RNN) network architecture.

FIG. 10B shows an example self-attention unit. The self-attention unit may be used in a self-attention network (SAN) network architecture.

Transfer learning may be implemented on the three aforementioned neural network architectures. In transfer learning, the training procedure of neural networks may include two steps: first, the networks may be pre-trained on other patients' data by excluding the data from the target patient, and then the network may be fine-tuned on one part of the target patient's data. The network may be tested on the rest of the data from the target patient. Two commonly-used further-training approaches are based on initialization and feature extraction. In the initialization approach, the entire network may be further trained, while in the feature extraction approach the last few fully-connected layers may be trained from a random initialization while other layers remain unchanged. The present methods and systems, in addition to these two approaches, may implement a third approach by combining these two approaches, i.e., the last few fully-connected layers are further trained while other layers remain unchanged. The various architectures and learning methods are summarized in Table 1 below:

| | Models | Details |
|---|---|---|
| Network | RNN | GRU size 10, 2 GRUs; FNN width 10, 1 FNN |
| architecture | | layer |
| | SAN | 8 self-attention units; FNN width 10, 4 FNN |
| | | layers |
| | CNN | kernel size 4, 4 conv seq2seq units; FNN width |
| | | 10, 3 FNN layers |
| Transfer | Transfer1 | Reuse weights of feature block and FNN block, |
| learning | | retrain both blocks |
| method | Transfer2 | Reuse weights of feature block and FNN block, |
| | | retrain FNN block |
| | Transfer3 | Reuse weights of feature block, reinitialize |
| | | FNN block, retrain FNN block |

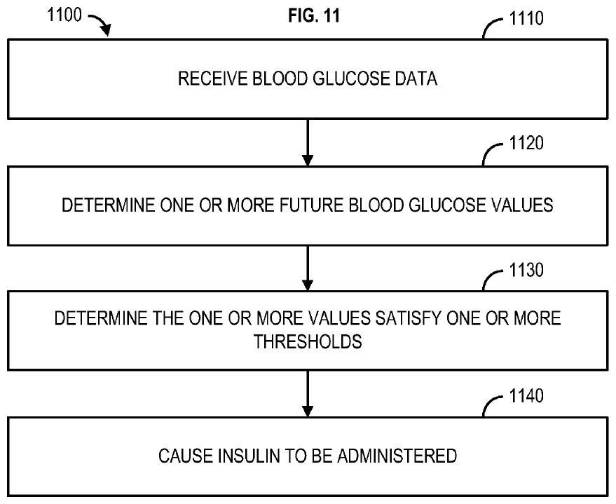

FIG. 11 shows an example method 1100 for preventing glycemic events. The method may comprise generating, training, and outputting one or more improved deep-learning models. The method 1100 may be performed in whole or in part by a single computing device, a plurality of computing devices, and the like. The method 1100 may be carried out (e.g., executed on) any one or more devices of the present disclosure or any combination thereof. The method may be carried out by system of devices. For example, the system may comprise a glucose sensor, a control algorithm, and an insulin infusion device. The system may be referred to as an artificial pancreas. The artificial pancreas (AP) is a closed-loop system designed for patients with T1DM to improve their BG regulation, and consequently decrease the risk of diabetic complications. The method 1100 may result in appropriate insulin treatment comprising dosing adjustments of insulin (e.g., exogenous insulin administered by a user and/or insulin administered by a device such as an insulin pump) to account for changes in one or more physiological parameters such as carbohydrates intake, physical exercise, and illness or stress.

At step 1110, one or more predictive models may receive current blood glucose data from a patient. The predictive model is associated with one or more ordinary differential equations (ODEs) and wherein one or more coefficients of the one or more ODEs are associated with one or more physiological parameters of the predictive model. The method may be executed on a variety of hardware and software components as described herein including various network structures and learning methods. The one or more predictive models may be trained on population data and patient data as described herein. The one or more predictive models may be trained so as to determine a likelihood of a glycemic event or other medical event. The glycemic event may comprise a glycemic episode wherein a blood glucose value associated with a patient falls below or rises above one or more blood glucose thresholds. For example, the glycemic event may comprise a hypoglycemic event wherein the blood glucose value associated with the patient falls below 70 mg/dL. For example, the glycemic event may comprise a hyperglycemic event wherein the blood glucose value associated with the patient rises above 120 mg/dL. The aforementioned ranges are merely exemplary and explanatory. The one or more blood glucose thresholds may be any value. Further, the method may comprise adjusting, on a per-patient basis, the one or more blood glucose thresholds. For example, the artificial pancreas may adjust the one or more thresholds based on patient-specific data gathered, for example, from a history of physiological parameter inputs. The artificial pancreas may comprise one or more offline reinforcement learning algorithms (e.g., batch constrained Q learning, or “BCQ”) where the parameters are inferred with system biology informed neural networks (SBINNs) and patient specific data. The population data may include medical data associated with a plurality of persons. For example, the population data may include one or more blood glucose histories of the plurality of persons. The one or more blood glucose histories may comprise, for each person of the plurality of persons, blood glucose levels and associated information such as temporal information (e.g., times and dates at which the one or more blood glucose levels for each person of the plurality of persons is determined), weight, carbohydrate intake, exercise, stress, levels interstitial blood glucose measures, plasma glucose measures, subcutaneous cell glucose measures, combinations thereof, or the like. Additional medical history data may also be included in the population data, such as for each person of the plurality of persons, demographic data such as age and gender, body composition data such as body mass, height, BMI and similar information, hormonal levels including cortisol, leptin, fasting glucose, insulin, HOMA 1-IR, and blood glucose data brief data such as data reading length (hours), model input blood glucose (BG) length (minutes), hypoglycemia threshold (mg/dL), hyperglycemia threshold (mg/dL), and HbA1c (%). Determining the population data may comprise receiving the population data from a database. For example, a database operated by a healthcare facility or network may be queried and in response to the query, the population data may be received. The population data may comprise a publicly available dataset including continuous glucose monitoring, insulin, physiological sensor, and self-reported life-event data

The current blood glucose data may comprise one or more blood glucose values. The one or more blood glucose values may be associated with time data such as a clock time and/or a time increment such as an hour or a minute. The time increment may comprise, for example, a time range such as five minutes. The patient specific data may be determined. The patient specific data may comprise one or more physiological parameters such as carbohydrates intake, physical exercise, and illness or stress). The neural network may comprise a convolutional neural network. The FNN may comprise a convolutional neural network. The convolutional neural network may be trained through backpropogation.

At step 1120, based on the current blood glucose data, one or more future blood glucose values for the patient may be determined. Determining the one or more future blood glucose values for the patient may comprise training the predictive model using previous blood glucose data from a population and training the predictive model using the previous blood glucose data from the patient, wherein the population blood glucose data has been augmented. The one or more future blood glucose values may comprise at least one predicted blood glucose value. The at one least one predicted blood glucose value may be a blood glucose value at point in time in the future for example from 5 minutes to 60 minutes in the future. Training the predictive model may comprise using previous blood glucose data from the patient. Training the predictive model may comprise fixing weights and biases in the feature block and tuning weights and biases in the FNN. The predictive model may be trained on the minority class. The minority class may comprise previous blood glucose data associated with values lower than 80 mg/dL. Modifying the previous blood glucose data to augment a minority class within the previous blood glucose data comprises generating synthetic minority samples. The synthetic minority samples are generated using TimeGAN or mixup.

At step 1130, it may be determined that the one or more future blood glucose values satisfy one or more thresholds. The one or more thresholds may comprise measures of insulin. It may be determined that the one or more future blood glucose values for the patient is indicative of a glycemic event. The determination may be based on the one or more future blood glucose values for the patient satisfying, or failing to satisfy a blood glucose value threshold. For example, a first threshold of the one or more thresholds may be 70 mg/dl of insulin and a second threshold of the one or more thresholds may be 120 mg/dl. The aforementioned are merely exemplary and explanatory and any value threshold may be used. Further, the one or more thresholds may be adjusted, or initial set to different values, based on training the patient specific predictive model. The blood glucose value threshold may comprise 70 mg/L, 120 mg/L, or the like. For example, if it is determined that a future blood glucose event falls below 70 mg/L, it may be determined that the future blood glucose event comprises a hypoglycemic event. For example, if it is determined that the future blood glucose event falls above 120 mg/L, it may be determined that the future blood glucose event comprises a hyperglycemic event. For example, if it is determined that the future blood glucose event falls between 70 mg/L and 120 mg/L, it may be determined that the future blood glucose event comprises a normoglycemic event.

At 1140, the method may cause an administration of insulin. For example, an insulin pump may be caused to administer insulin. For example, the method may cause a user device to display a prompt configured to prompt a user to administer insulin. The method may comprise determining, based on current BG data, one or more physiological parameters such as carbohydrate intake, exercise, sleep, stress, or the like, and known insulin dynamics for the patient, an amount of insulin configured to prevent the occurrence of the predicted glycemic event.

The method may comprise sending, based on the determination as to whether the one or more future blood glucose events comprise a hypoglycemic event, a normoglycemic event, or a hyperglycemic event, a message. The message may comprise an alarm, an alert, combinations thereof, and the like.

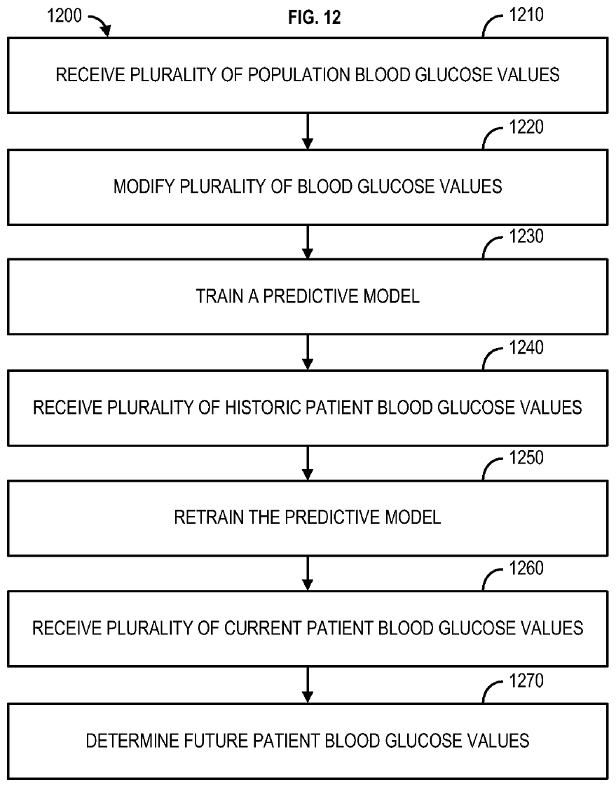

FIG. 12 shows an example method 1200 for predicting future blood glucose values of a patient. The method 1200 may be performed in whole or in part by a single computing device, a plurality of computing devices, and the like.

At step 1210, a plurality of blood glucose values may be received from a population of individuals. Receiving the blood glucose values may comprise receiving the plurality of blood glucose values from a database. For example, a computing device may query the database and in response to the query, receive the plurality of blood glucose values from the population of individuals. The plurality of current blood glucose values may comprise one or more blood glucose values, wherein each of the one or more blood glucose values is associated with a time increment. The time increment may be any length of time, for example, 5 minutes. The predictive model may comprise a neural network comprising a feature block and a feed-forward neural network (FNN). The feature block may comprise a CNN.

At step 1220, the plurality of blood glucose values from the population of individuals may be modified. Modifying the plurality of blood glucose values from the population of individuals may comprise performing, on the plurality of blood glucose values from the population of individuals, one or more data augmentation methods. For example, the one or more data augmentation methods may comprise one or more of oversampling by repeating, adding Gaussian noise, performing a mixup method, or utilizing TimeGAN as described herein. Performing the one or more data augmentation methods may result in an augmented dataset. The augmented dataset may comprise the plurality of blood glucose values from the population of individuals, as well as an augmented minority class. The minority class may comprise blood glucose values associated with one or more of a hypoglycemic label or a hyperglycemic label (as opposed to, for example, a normoglycemic label or euglycemic label), and thus, the augmented dataset may include more minority samples than the un-augmented plurality of blood glucose values from the population of individuals. The minority class may comprise blood glucose values lower than 80 mg/dL and/or blood glucose values higher than 180 mg/dL. Modifying the previous blood glucose data to augment a minority class within the previous blood glucose data may comprise generating synthetic minority samples. The synthetic minority samples be generated using oversampling by repeating, adding Gaussian noise, TimeGAN, mixup, or any other suitable data augmentation.

At step 1230, a predictive model may be trained using the modified (e.g., augmented) plurality of blood glucose values. The predictive model may comprise the population level model described herein. Training the predictive model using previous blood glucose data from the patient further may comprise fixing weights and biases in the feature block and tuning weights and biases in the FNN.

At step 1240, a plurality of previous blood glucose values may be received from a patient. Receiving the plurality of previous blood glucose values from the patient may comprise the computing device querying a database and, in response to the query, the database may send to the computing device, the plurality of previous blood glucose values from the patient. The plurality of previous blood glucose values from the patient may be divided into a training part and a testing part as described herein. For example, the training part may comprise a percentage or number of values of the plurality of previous blood glucose values from the patient and the testing part may comprise a remaining percentage or number of values of the previous blood glucose values from the patient.

At step 1250, the predictive model may be trained (e.g., retrained) using the plurality of previous blood glucose values from the patient. Training (e.g., retraining) the predictive model using the plurality of previous blood glucose values from the patient may comprise implementing one or more of, and/or a combination of the transfer learning methods described herein including, but not limited to, Transfer1, Transfer2, and/or Transfer 3.

At step 1260, a plurality of current blood glucose levels from the patient may be received. The plurality of current blood glucose levels from the patient may be received, for example, from a blood glucose monitoring device such as the wearable device 503C and/or the pump/monitor device 505. The plurality of current blood glucose levels from the patient may comprise one or more discrete blood glucose measurements and/or a continuous stream of blood glucose measurements

At step 1270, one or more future blood glucose values for the patient may be determined. The one or more future blood glucose values for the patient may comprise one or more predicted blood glucose values for the patient. Determining the one or more future blood glucose values for the patient may comprise predicting, via the predictive model, the one or more future blood glucose values for the patient. For example, the one or more current blood glucose values for the patient may be received by the trained (e.g., retrained) predictive model as an input and the trained predictive model may output, based on the input, the one or more future blood glucose values for the patient. The one or more future blood glucose values may comprise at least one blood glucose value from 5-60 minutes in the future.

The method 1200 may further comprise determining whether the one or more future blood glucose values comprises a hypoglycemic event.

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of the methods and systems. Efforts have been made to ensure accuracy with respect to numbers (e.g., amounts, temperature, etc.), but some errors and deviations should be accounted for. Unless indicated otherwise, parts are parts by weight, temperature is in C or is at ambient temperature, and pressure is at or near atmospheric.

### Example 1

In the following examples, the details of the classification tasks are introduced, and then the methods of deep transfer learning and various data augmentation techniques for overcoming the challenges of small datasets and imbalanced data are presented.

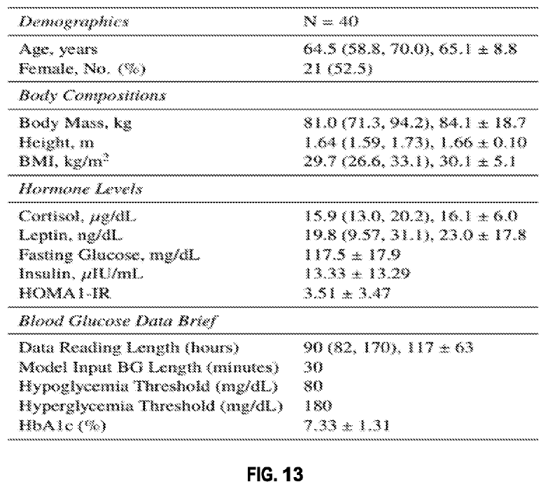

The following examples may refer to baseline characteristics, baseline data, or population data. Baseline characteristics are indicated in FIG. 13. Baseline characteristics were obtained from 40 patients with diabetes (19 males, mean [SD], age 65 [8] years) was analyzed. All level 1 hypoglycemic and hyperglycemic episodes from the CGM recordings were identified. A selection of the baseline characteristics of the patient cohort is reported in FIG. 13.

### Patient-Specific Prediction of Blood Glucose

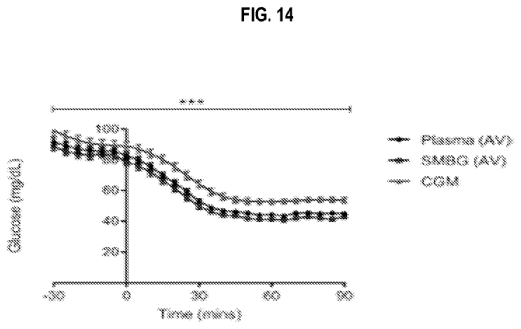

The following two classification tasks of diabetic blood glucose, e.g., one classification is “hypoglycemia” vs. “no hypoglycemia” and the other is “hypoglycemia” vs. “normoglycemia” vs. “hyperglycemia”, were considered with the setup shown in FIG. 7A. Specifically, the threshold for hyperglycemia is set to 180 mg/dL, e.g., blood glucose levels higher than 180 mg/dL is labeled with “hyperglycemia”. On the other hand, the threshold for hypoglycemia is to be 80 mg/dL, (e.g., blood glucose levels lower than 80 mg/dL are labeled as “hypoglycemia”). Here, unlike the common definition for level 1 hypoglycemia based on the threshold of 70 mg/dL, 80 mg/dL was chosen as the hypoglycemia threshold. This is because recent results have revealed a measurement artifact, e.g., that the real-time Continuous Glucose Monitoring (CGM), where one would expect these algorithms to have clinical applicability, underestimates the degree of hypoglycemia by a difference of 10 mg/dL, as shown in FIG. 14.

### Deep Transfer Learning for Small Patient-Specific Data

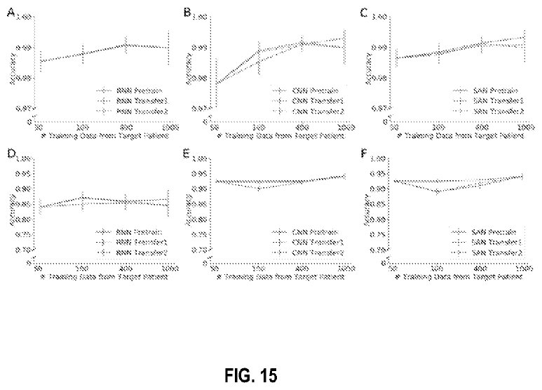

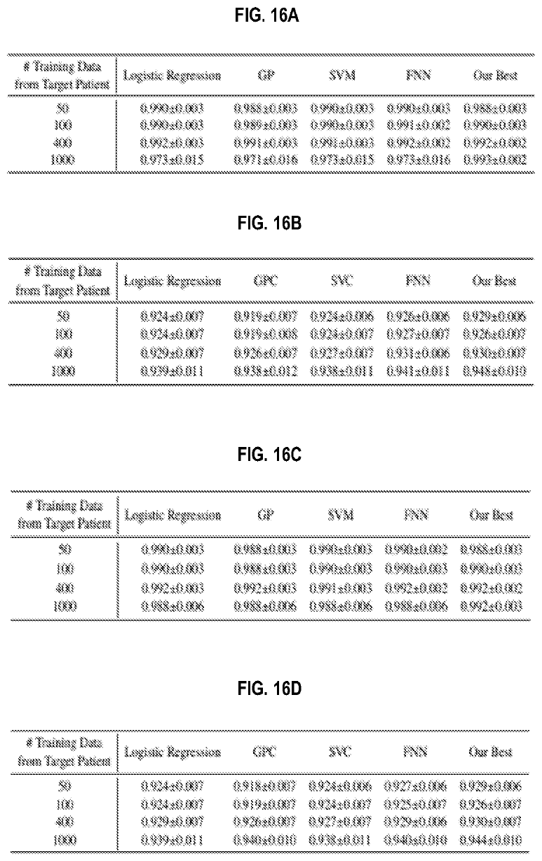

The performance of three neural network architectures by the averaged prediction accuracy per capita for these two classification problems were compared. The results in FIG. 15 indicate that as the training data size from the target patient increases, the prediction accuracy of all models generally increases. It was noted that CNN models are generally more accurate than RNN models and slightly outperform SAN models with higher mean and smaller standard deviation for prediction accuracy in both of the classification tasks. FIG. 15 shows an example prediction accuracy comparison among different network architectures (RNN, CNN, and SAN). A to C show prediction accuracy of the binary classification, (e.g., identifying the neural network output as hypoglycemia or no hypoglycemia), using (A) RNN, (B) CNN and (C) SAN. D to F show prediction accuracy of the three-class classification, e.g., identifying the neural network output as hypoglycemia or normoglycemia or hyperglycemia, using (D) RNN, (E) CNN and (F) SAN. The data from a target patient may be divided into two parts, one is for training and the other is for testing, and the prediction horizon was fixed at 30 minutes. The transfer learning may consist of two-step training. In step 1, models were trained with the data from all the patients except for this target patient; in step 2, the models obtained from step 1 were re-trained with the training data from this target patient. Hence, the training data size (number of training data) from the target patient is an important factor controlling the prediction accuracy. Transfer3 exhibited poor results while Transfer2 exhibited comparatively good results. The results also indicate that the transfer learning models (Transfer1 and Transfer2) can sometimes outperform the pre-trained models in CNN models. The presently described models were compared with some existing classification methods, (e.g., logistic regression, GP, SVM and FNN) in terms of 1) Predicting hypoglycemia vs. no hypoglycemia (FIG. 16A); 2) Predicting hypoglycemia vs. normoglycemia vs. hyperglycemia (FIG. 16B) over a prediction horizon of 30 minutes; 3) Predicting hypoglycemia vs. no hypoglycemia (FIG. 16C); 4) Predicting hypoglycemia vs. normoglycemia vs. hyperglycemia (FIG. 16D) over a prediction horizon of 60 minutes. FIG. 16A shows Hypoglycemia detection (binary classification, hypoglycemia vs. no hypoglycemia) accuracy (mean±standard error) of different methods with different numbers of training data from the target patient, for prediction horizon at 30 minutes and training models on the cohort (patients participated in this study) data. GP, Gaussian Process; SVM, supporting vector machine; FNN, the best fully-connected neural networks; the Best, the best model among the combinations of three proposed architectures (RNN, CNN and SAN) and four transfer learning methods (Pretrain, Transfer1, Transfer2 and Transfer3). FIG. 16B shows hypoglycemia and hyperglycemia detection (three-class classification) accuracy (mean±standard error) of different methods with different numbers of training data from the target patient, for prediction horizon at 30 minutes and training models on the cohort (patients participated in this study) data. In three-class classification, the output label of prediction is one out of three labels, “hypoglycemia”, “normoglycemia” and “hyperglycemia”, at any given time. GP, Gaussian Process; SVM, supporting vector machine; FNN, the best fully-connected neural networks. Our Best, the best model among the combinations of three proposed architectures (RNN, CNN and SAN) and four transfer learning methods (Pretrain, Transfer1, Transfer2 and Transfer3). FIG. 16C shows Hypoglycemia detection (binary classification) accuracy (mean±standard error) of different methods with different numbers of training data from the target patient, for prediction horizon at 60 minutes. GP, Gaussian Process; SVM, supporting vector machine; FNN, the best fully-connected neural networks; Our Best, the best model among the combinations of three proposed architectures (RNN, CNN and SAN) and four transfer learning methods (Pretrain, Transfer1, Transfer2 and Transfer3). FIG. 16D shows Hypoglycemia and hyperglycemia detection (three-class classification) accuracy (mean±standard error) of different methods with different numbers of training data from the target patient, for prediction horizon at 60 minutes. GP, Gaussian Process; SVM, supporting vector machine; FNN, the best fully-connected neural networks; Our Best, the best model among the combinations of three proposed architectures (RNN, CNN and SAN) and four transfer learning methods (Pretrain, Transfer1, Transfer2 and Transfer3). In both tasks, the present models showed consistent increases in accuracy and the area under the receiver operating characteristic curve (AUROC) given more training data from the target patient and, specifically, better than those by existing classification methods examined in predicting hypoglycemia vs. normoglycemia vs. hyperglycemia.

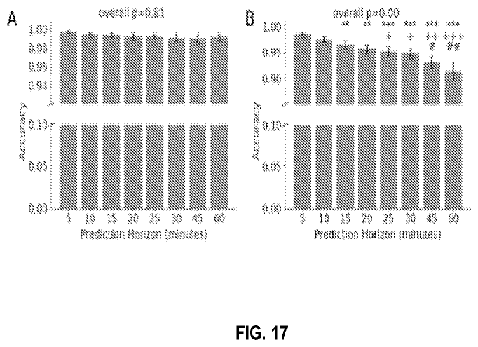

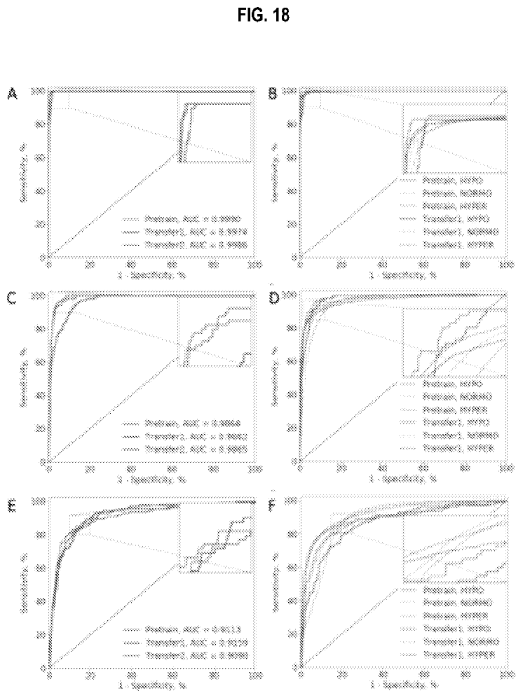

FIG. 17 shows the sensitivity analysis of the prediction horizon on the prediction accuracy and FIG. 18 shows the ROC curves (receiver operating characteristic curves) of best models among all the models tested, given the training data size from the target patient around 1000 data segments. The sensitivity between different prediction horizons is negligible in predicting hypoglycemia vs. no hypoglycemia (binary classification), while the sensitivity between different prediction horizons becomes larger when the time elapse of two prediction horizons is large in predicting hypoglycemia vs. normoglycemia vs. hyperglycemia (three-class classification). FIG. 17 shows the prediction accuracy for two classification tasks given different prediction horizons using the best CNN model. Prediction accuracy for (A) binary classification, e.g., identifying the neural network output as hypoglycemia or no hypoglycemia, and (B) three-class classification, e.g., identifying the neural network output as hypoglycemia or normoglycemia or hypoglycemia, given different prediction horizons. No statistical significance is observed for binary classification. *, p-value≤0.05; **, p-value≤0.01; ***, p-value≤0.001, in comparison to the prediction horizon at 5 minutes; +, p-value≤0.05; ++, p-value≤0.01; +++, p-value≤0.001, in comparison to the prediction horizon at 10 minutes and #, p-value≤0.05; ##, p-value≤0.01; ###, p-value≤0.001, in comparison to the prediction horizon at 15 minutes.

FIG. 18 shows ROC curves for two classification tasks given prediction horizons at 5 minutes, 30 minutes and 60 minutes using the best CNN model. (A, B) Examples of the ROC curves for the prediction horizon at 5 minutes, in (A) binary classification and (B) three-class classification. (C, D) Examples of the ROC curves for the prediction horizon at 30 minutes, in (C) binary classification and (D) three-class classification. (E, F) Examples of the ROC curves for the prediction horizon at 60 minutes, in (E) binary classification and (F) three-class classification. AUC, area under the ROC curve. Binary classification denotes predicting hypoglycemia vs. no hypoglycemia. In three-class classification, the labels (HYPO for “hypoglycemia”, NORMO for “normoglycemia” and HYPER for “hyperglycemia”) were iterated over to compute the ROC curves. For example, one of the labels, “hypoglycemia”, was taken as the TRUE label and treated the rest of the labels, e.g., “normoglycemia” and “hyperglycemia”, together as the FALSE label to generate the ROC curves. The results by Transfer2 is comparable to those by Pretrain while those by Transfer3 are worse than Transfer1 and Transfer2, hence only the results of Pretrain and Transfer1 are shown for brevity. Details of the transfer learning methods can be found in FIGS. 7B-10B and Table 1.

### Improvement of Sensitivity for Imbalanced Data

In this section, further detailed analysis is shown with regression based models for classification, e.g., regression prediction was performed then the real-valued prediction was converted into class labels, as shown in FIG. 7A. It is noted that raw BG data is innately real-valued, hence it is natural to investigate the data feature following a regression approach. Here, the aim is to show the effects of different data augmentation methods mainly on the minority dataset. With previous classification analysis, the regression model was set up with the following preconditions: the prediction horizon is 20 minutes if not mentioned otherwise and the hypoglycemia threshold is set at 80 mg/dL. Results are shown without transfer learning, e.g., the models are trained on the dataset, which is the union of other patients' data except for the target patient and then directly test on the target patient's dataset. The focus was on comparing the model performance in predicting hypoglycemia vs. no hypoglycemia by converting the real-valued prediction into two labels: one label is “hypoglycemia”, meaning the prediction is below 80 mg/dL while the other is “no hypoglycemia”, meaning the prediction is above or equal to 80 mg/dL. The same conversion was carried out on the true BG values measured by the CGM. With the conversion, it is possible to compare four classification scores, sensitivity, positive predictive value, specificity and negative predictive value between different models.

### Selection of Loss Function

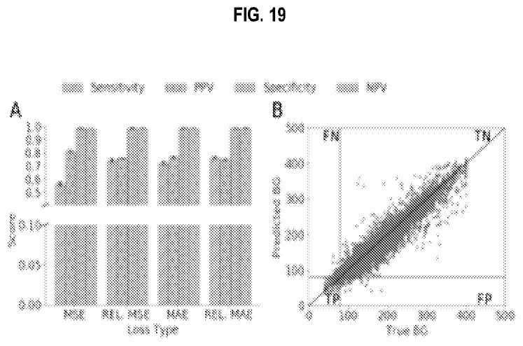

Four different loss functions were tested (e.g., mean absolute error, relative mean absolute error, mean squared error and relative mean squared error) using the original training dataset without data augmentation. In particular, the performance of models with different loss functions using four classification metrics, e.g., sensitivity, positive predictive value (PPV), specificity and negative predictive value (NPV) were examined. FIG. 19 shows the comparison of model performance using different loss functions. The result suggests that the model using relative mean absolute error (REL. MAE) outperforms models using the other three loss functions, because the model using the relative mean absolute error maintains a balanced high value for each of the aforementioned four metrics. The scattering plot of true BG vs. predicted BG also suggests high prediction accuracy with the points clustering near the diagonal black line indicating the perfect prediction.

### Data Augmentation

In this part, the loss function was fixed to be the relative mean absolute error (REL. MAE) and a comparison was made of the performance of the present model when four different data pre-processing techniques are implemented for data augmentation on the training data of the minority class.

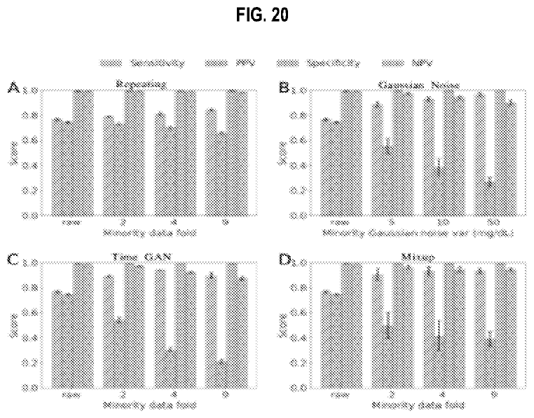

Oversampling by repeating. For this data augmentation method, minority samples (the input-output pairs where the output BG is less than 80 mg/dL) were repeated in the training dataset for k folds, (e.g., for 2-fold oversampling by repeating), the minority samples were duplicated once such that the minority data is doubled in the augmented training dataset. Hence, for k-fold oversampling by repeating, the training data was augmented by adding k−1 copies of the training data labeled as hypoglycemia (output BG less than 80 mg/dL) to the augmented training dataset. FIG. 20B is shown as denoting that the true BG is “hypoglycemia” but the prediction is “no hypoglycemia”; the true negative region (TN) denoting that the true BG is “no hypoglycemia” and the prediction is “no hypoglycemia”.

Gaussian noise. Adding Gaussian white noises to the training dataset has been proved to be an effective way of data augmentation for CNNs, and specifically for CNNs using wearable sensor data. Different levels of Gaussian white noises distinguished by the variance of the noise were implemented. In particular, noise with variance was infused at 5, 10, 50 mg/dL to the input BG data of minority class, whose output BG value is below the hypoglycemia threshold, (e.g., there are two copies of minority training data in the augmented dataset, one is the original copy collected by the CGMs, and the other is a copy generated by infusing Gaussian noises).

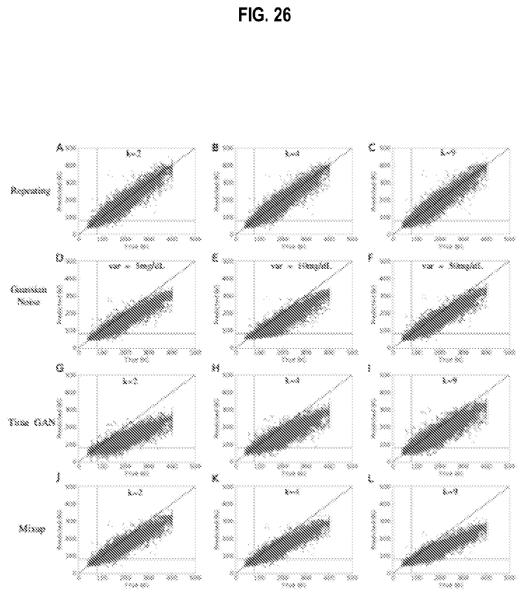

Time GAN. Synthetic minority samples were generated using Time GAN on the data of minority class and compared the performance of models when different folds of synthetic data of minority class were added to augmented dataset in the beginning of each training epoch. The GAN model generated blood glucose sequences with hypoglycemia labels very similar to the true blood glucose sequences fed into the model, suggested by the PCA and T-NSE plots for the original data and synthetic data, see FIG. 26. FIG. 20C shows that adding more minority data generated by Time GAN also improves model sensitivity but not as monotonically as the other methods tested.

Mixup. The generalization of neural network architectures may be improved by linearly interpolating between samples in the training dataset using the following formula, \tilde{x}=λxᵢ+(1−λ)xⱼ, \tilde{y}=λyᵢ+(1−λ))yⱼ, where x^{˜}, y^{˜} denote generated input and output, respectively; A is a hyperparameter following the Beta distribution, Beta(α, α); xᵢ, xⱼ denote inputs from two different samples and yᵢ, yⱼ denote the corresponding output of those two different samples. It is noted that in the original mixup algorithm, yᵢ, yⱼ can be of different class, while in the present case only mixup was performed on the minority class, e.g., yᵢ, yⱼ satisfy the condition that yᵢ<80 and yⱼ<80.

There have been some attempts to perform data augmentation using mixup in time-series analysis of biosignals, such as electroencephalogram (EEG) and electrocardiogram (ECG), generating virtual biosignals from real biosignals of different types. The present methods and systems are the first to implement mixup for data augmentation on minority class only to alleviate the effect of data imbalance as well as the first to implement k-fold mixup. By k-fold mixup, the size of the minority class was increased to k times of its original size by adding k−1 copies of synthetic data using mixup for each training epoch. The original mixup algorithm does not include k as a hyperparameter, e.g., in the original mixup, the original training data is replaced by synthetic data generated by linear interpolation in the beginning of each training epoch. FIG. 20D) shows that increasing the folds of minority data by mixup could help improve model sensitivity but the uncertainty in the positive predictive value is relatively larger than other augmentation methods.

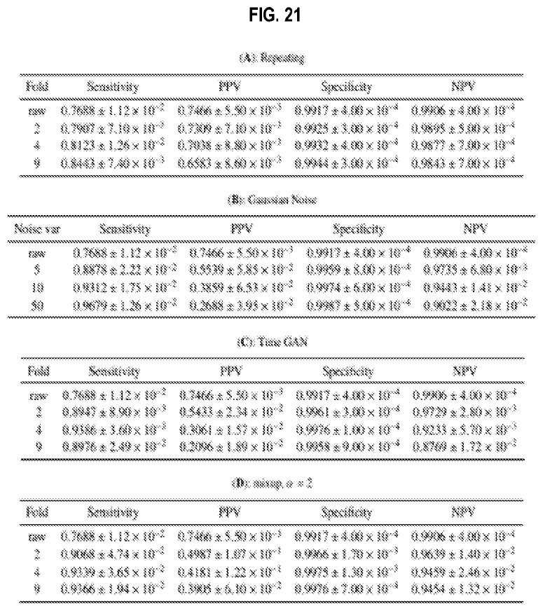

FIG. 21 shows a comparison of four classification scores using four different data augmentation methods on minority class. Classification scores for model trained on dataset with (A) repeating, (B) Gaussian noise, (C) Time GAN (D) mixup (α=2). PPV, positive predictive value. “Raw,” model without data augmentation. NPV, negative predictive value. Noise Var, noise variance. Positive class is the class of samples labeled with hypoglycemia, which is also the minority class.

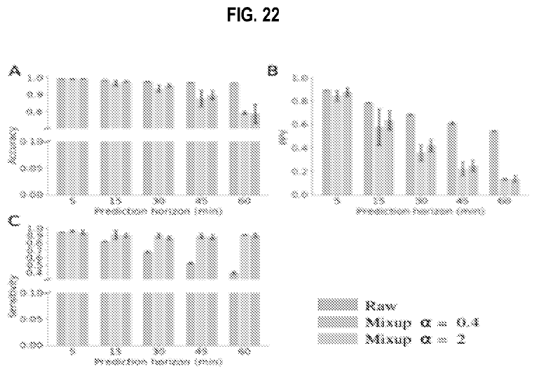

The hyper-parameter α in the Beta distribution Beta(α, α) of mixup is A parameter controlling the diversity of the synthetic samples, (e.g., higher α produces samples more resembling to the reference real data while lower α introduces samples very different from the reference real data). With α=1, Beta(1, 1) is equivalent to a uniform random distribution. The performance of the present model given α=0.4 was compared with the present model given α=2 in 2-fold mixup, in terms of two classification scores, e.g., positive predictive value (PPV) and sensitivity for the positive class (the minority class, hypoglycemia samples), and the sensitivity of those two classification scores for different prediction horizons was examined. The results for α=0.4 are shown in FIG. 22A and those for α=2 in FIG. 22B. It is noted that mixup with either α=0.4 or α=2 improves the model sensitivity over different prediction horizons. Specifically, models trained on the training dataset augmented by mixup show high sensitivity within all the prediction horizons examined while the model without data pre-processing shows decreased sensitivity over longer prediction horizons. The model trained on the training dataset augmented by mixup α=0.4 shows different uncertainty in the predictive scores for different prediction horizons; for example, the standard deviation of sensitivity and PPV for prediction horizon at 15 minutes are much larger than those for other prediction horizons. However, the model trained on the training dataset augmented by mixup α=2 shows similar uncertainty in the predictive scores among different prediction horizons, mainly because samples generated by mixup α=0.4 are relatively distinct from the original samples collected while those by mixup α=2 is similar to the original samples, hence preserves the data patterns.

The results in FIG. 20 indicate that by adding more training data of minority class, either through duplication or synthesizing, will increase the model sensitivity but decrease the positive predictive value, e.g., the precision for minority class. Specifically, given the same amount of minority samples in the training data, the increase in model sensitivity and decrease in precision for minority class is more significant in those with synthetic minority samples, compared to the oversampling by repeating. These results prove a recent finding that transforms (augmentations) which preserve the labels of the data can improve estimation by enlarging the span of the training data. In the present case, the labels of the data are preserved by only augmenting the minority training data, which consequently increases the span of minority data, by generating synthetic data using Gaussian noise, Time GAN or mixup. Results also suggest that synthetic minority data (data generated by infusing Gaussian noise, Time GAN or mixup) could increase the span of minority data much more significantly than repeating the original minority data.

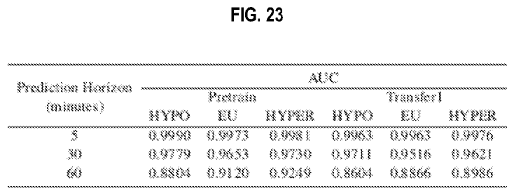

FIG. 23 shows hypoglycemia and hyperglycemia detection Area Under ROC (AUC) summary. The scores are summarized from FIG. 17.

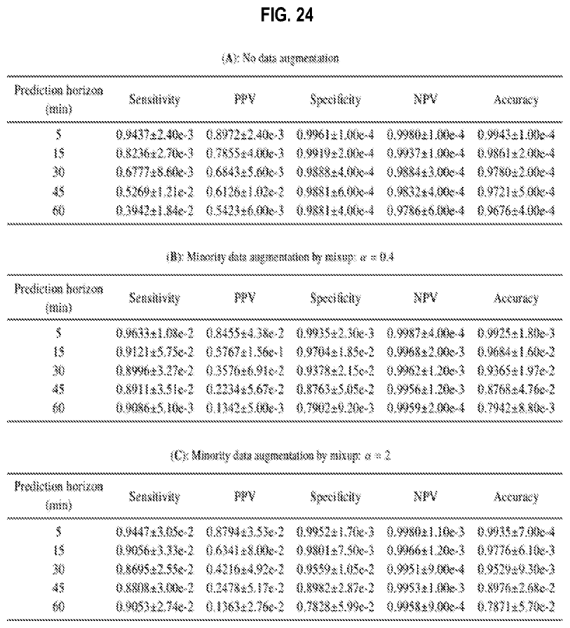

FIG. 24 shows a comparison of three classification scores for minority class between two mixup models and the original dataset. Classification scores for model trained on dataset with (A) no data augmentation, (B) minority data augmentation by mixup (α=0.4) and (C) minority data augmentation by mixup (α=2). PPV, positive predictive value. NPV, negative predictive value. Positive class is the class of samples labeled with hypoglycemia, which is also the minority class.

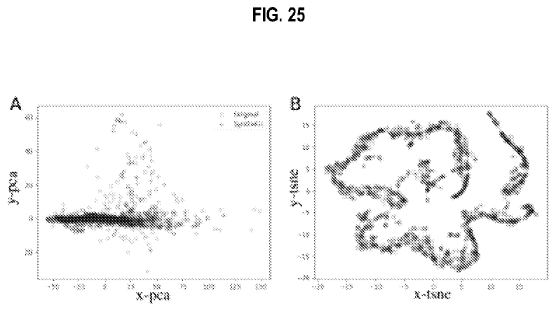

FIG. 25 shows the visualization results of the hypoglycemia samples generated by Time GAN (A) PCA (principle component analysis) and (B) T-SNE. The discriminative score output from the trained Time GAN is around 0.10.

FIG. 26 shows true BG measured by CGM vs. BG prediction from the model trained with data augmentation on the minority training data, given different data augmentation/preprocessing methods. Minority training data is augmented with (A to C) oversampling by repeating, (D to F) Gaussian white noise infusion to a copy of minority training data, (G to I) Time GAN and (J to L) mixup. Minority training data size is (A, G, J) doubled or (B, E, K) becomes 4 times or (C, I, L) becomes 9 times of its original size after data augmentation. Minority training data size is doubled in (D to F), but different noise level is considered in each figure, e.g., the new copy of minority data is generated by infusing Gaussian white noise with (D) variance 5 mg/dL, (E) variance 10 mg/dL and (F) variance 50 mg/dL to the original minority training data. It is noted that with data augmentation methods, such as Gaussian noise, Time GAN and mixup, which introduce synthetic data, the model sensitivity increases to more than 90%. Specifically, for Time GAN and mixup, increasing the minority data fold k, further increases the model sensitivity. However, the increase in sensitivity sacrifices the precision of the minority class (hypoglycemia), as indicated by the inclination of the scattering points, e.g., the (True BG, Predicted BG) pairs, towards x axis shown in the FIG. 26.

### Discussion

Type 2 diabetes is considered an epidemic worldwide. Hyperglycemia selectively damages cells that are not able to reduce glucose transport into the cell, such as capillary endothelial cells in the retina, mesangial cells in the renal glomerulus, and neurons and Schwann cells in peripheral nerves. High intracellular glucose concentration leads to the exhaustion of the antioxidant pathways, altered regulation of gene transcription and increased expression of pro-inflammatory molecules resulting in cellular dysfunction and death. On a clinical level, these cellular changes translate into micro and macrovascular complications of diabetes associated with poor outcomes and increased mortality. Current diabetes treatment regimens may decrease the occurrence of complications associated with hyperglycemia, however, they also suppose a risk of extremely low glucose levels. Hypoglycemia can lead to permanent neurological damages if not treated promptly and increased mortality. The prediction of blood glucose variations helps to adjust acute therapeutic measures and food intake in patients with type 2 diabetes.

Transfer learning methods were developed to predict hypoglycemia” vs. “no hypoglycemia” or “hypoglycemia” vs. “normoglycemia” vs. “hyperglycemia” for patients with type 2 diabetes. State-of-the-art results were obtained by tackling two major challenges associated with the small data size for individual patients as well as the imbalanced datasets, (e.g., small samples for hypoglycemia). To deal with small datasets, three neural network models were considered, including recurrent neural networks (RNNs), convolutional neural networks (CNNs) and self-attention networks (SANs). Also examined were four transfer learning strategies, which enabled training the neural networks with a small amount of individual's recorded data. The performance of the method was demonstrated on the data obtained from 40 patients. High prediction accuracy was achieved for the task of predicting hypoglycemia vs. no hypoglycemia with accuracy no less than 98% and AUROC greater than 0.9 for all the prediction horizons examined. For the task of predicting hypoglycemia vs. normoglycemia vs. hyperglycemia, the best model among all tested models achieved high accuracy greater than 89% and AUROC greater than 0.86, for all the prediction horizons examined (up to one hour). Results indicate that as the prediction horizon prolongs, the prediction accuracy as well as the AUROC decreases, as expected, in both classification tasks.

When comparing the model performance on predicting hypoglycemia vs. no hypoglycemia and predicting hypoglycemia vs. normoglycemia vs. hyperglycemia, results indicate that the overall prediction accuracy and AUROC in the task of predicting hypoglycemia vs. no hypoglycemia is always higher than those in the task of predicting hypoglycemia vs. normoglycemia vs. hyperglycemia. More specifically, statistical significance was observed between two short prediction horizons (5 mins and 10 mins) and the largest prediction horizon (60 mins) in the task of predicting hypoglycemia vs. normoglycemia vs. hyperglycemia. It is noted that despite of the statistical differences observed among different prediction horizons, the model always maintained high accuracy.

However, a closer examination on the dataset reveals that most of the blood glucose levels are labeled as either normoglycemia or hyperglycemia and hence only very few blood glucose levels are labeled as hypoglycemia, making hypoglycemia the definite minority class, resulting in models with sensitivity around 77% and positive predictive value around 75% for a prediction horizon of 20 minutes. Given the need to detect hypoglycemia more accurately and robustly, data augmentation on the minority class, (e.g., augment the hypoglycemia samples in the training dataset), is an effective way of enforcing the neural network to learn the underlying patterns of the hypoglycemia data at a finer scale compared to learning on the dataset without data augmentation. Tests indicate that data augmentation on the minority class using synthetic data (not oversampling by repeating) increases the model sensitivity in detecting hypoglycemia, from less than 80% to more than 96% depending on the specific augmentation method for a prediction horizon of 20 minutes. This allows early treatment intervention and prevention of potential hypoglycemic events and hence is a significant improvement preferred in clinical diagnosis given the fatal consequences of hypoglycemia for patients with serious complications caused by type 2 diabetes.

However, given the imbalance nature of the training dataset, the increased sensitivity, (e.g., the recall of the minority class), observed from models trained on the augmented dataset also comes with a decrease in the positive predictive value, e.g., the precision of the minority class. Although the trade-off between the precision and recall for imbalanced datasets is a commonly observed dilemma, with minority data augmentation of different folds, it could still achieve a good balance between those two metrics such that they are acceptable in practical scenarios.

This method may be purely data-driven with no physiological knowledge, and performs prediction merely based on the blood glucose history. Data-driven methods relieve physicians from exhausting all possible combinations of physiological inputs given large samples or data. It is not an easy task to incorporate domain knowledge to data-driven methods, especially in neural network based models. In the disclosed methods, nutritional intake, exercise or stress conditions in dysglycemia prediction were identified as the domain knowledge, the appropriate incorporation of which could possibly improve the model accuracy. Hence, disclosed herein is the development of physiologics-informed neural network models. This method has important clinical implications in terms of preventing and avoiding this potentially lethal complication, e.g., through alerts generated directly to the patient or by linking the prediction algorithms to the programmable insulin pumps.

To summarize, a new method for predicting hypoglycemia vs. no hypoglycemia and predicting hypoglycemia vs. normoglycemia vs. hyperglycemia was proposed, and the method shows remarkable performance characterized by high prediction accuracy and AUROC as well as other metrics, including specificity and sensitivity. In particular, a combined approach of transfer learning and data augmentation for imbalanced data can be proved a very powerful new framework for short term predictions for type 2 diabetes. Here, the focus was one on time periods up to 60 minutes, with a notable sensitivity and positive predictive value of the model observed during the first 15 and 30 minutes. It is believed that accurate hypoglycemia prediction over this period of time offers the most in terms of having potential warning signs and preventing adverse events by hypoglycemia. By incorporating transfer learning, this method could provide patient-specific results in both predicting hypoglycemia vs. no hypoglycemia and predicting hypoglycemia vs. normoglycemia vs. hyperglycemia with relatively few blood glucose samples. For example, in the present case, 1000 time segments were used for a total of 83 hours from the target patient.

### Research and Methods

Deep learning algorithms for patient-specific blood glucose level prediction were developed. Three different neural network architectures, including recurrent neural networks, self-attention networks, and convolutional neural networks, and four different transfer learning strategies were considered. Logistic regression, Gaussian process (GP), fully-connected feedforward neural networks (FNN), and support vector machines (SVM) were implemented as the baseline models.

### Dataset

The use of blood glucose (BG) history of patients with T2D in this study were approved by the institutional review board (IRB) of the Beth Israel Deaconess Medical Center. The BG level was measured every 5 minutes by a Continuous Glucose Monitoring System. Data obtained from 40 outpatients with diabetes (19 males; age 65±8 years; BMI at 30±5; with a mean HbA1c level at 7.33%), who contributed a mean of 130.6 mg/dL blood glucose level through CGM (BG ranging from 40 mg/dL to 400 mg/dL) were analyzed. Individuals were eligible for inclusion if they were adults with a diagnosis of T2D patients using CGM. 10 patients (25% of the participants) were treated with insulin while 27 (67.5% of the participants) were receiving oral or (non-insulin) injectable antidiabetic drugs. The rest of the patients (3 patients, 7.5% of the participants) were treated without oral nor insulin medications. All level 1 hypoglycemic (BG level less than 80 mg/dL) and hyperglycemic (BG level greater than 180 mg/dL) episodes from the CGM recordings were identified. To facilitate the network training, the BG levels were scaled by 0.01, and a smoothing step on the BG measurements was applied to remove any large spikes that may be caused by patient movement. An overview of the dataset used in this work can be found in FIG. 13.

### Predictors and Outcomes

The primary outcome of interest in this study is the BG values in the future, e.g., 30 min later. BG measured in 30 minutes (7 BG values) are one input data segment and predict the future BG level after a prediction horizon, a time period from the most recent CGM measurement in the input BG values, as shown in FIG. 7A.

### Neural Network Architectures

In the present example, three network architectures were employed, including recurrent neural networks (RNNs), gated convolutional neural networks (CNNs) and self-attention networks.

Typically, the dominant deep learning method used for sequence learning is the RNN, which is a class of neural networks that allows previous outputs to be used as the inputs of the current step. The cell units in RNNs are usually chosen as long short-term memory units (LSTM) and gated recurrent units (GRU), which deal with the vanishing gradient problem encountered by traditional RNNs. In addition to RNNs, CNNs and self-attention networks have been proposed recently for time series forecasting, and achieved better performance than RNNs in certain tasks. In gated CNNs, convolutional kernels create hierarchical representations over the input time series, in which nearby BG measurements interact at lower layers while distant BG measurements interact at higher layers. The mechanism of attention was first proposed for machine translation, and it has been shown that the network architecture based solely on self-attention mechanism can also be used successfully to compute a representation of the sequence. Self-attention is an attention mechanism to compute a representation of the sequence by relating different positions of a sequence. In RNNs, the input sequence is fed into the network sequentially, while in CNNs and self-attention networks, the input sequence is fed into the network simultaneously, and thus an embedding of the position of input elements is required. For the hyperparameters in the networks, (e.g., depth and width), a grid search was performed to obtain an optimal set of hyperparameters. Details of the network architectures used in this study can be found in the FIGS. 7B-10B and Table 1.

### Transfer Learning

To address the difficulty of obtaining a sufficient large dataset for each patient, implemented transfer learning was implemented on the three aforementioned neural network architectures. In transfer learning, the training procedure of neural networks includes two steps: first, the networks are pre-trained on other patients' data by excluding the data from the target patient, and then the network is further fine-tuned on one part of the target patient's data. Finally, the network is tested on the rest of the data from the target patient. Two commonly-used further-training approaches are based on initialization and feature extraction. In the initialization approach, the entire network is further trained, while in the feature extraction approach the last few fully-connected layers are trained from a random initialization while other layers remain unchanged. In this study, in addition to these two approaches, a third approach was implemented by combining these two approaches, e.g., the last few fully-connected layers are further trained while other layers remain unchanged. The details of the four transfer learning methods can be found in FIGS. 7B-10B and Table 1.

### Imbalanced Data

Imbalanced data has been a ubiquitous issue in many fields, causing most methods to yield erroneous predictions strongly biasing towards the majority class. To reduce the hazardous effect of imbalanced data, the method can be improved with various techniques: (i) modifying the imbalanced data set by some mechanisms such as oversampling or undersampling or both to provide a balanced distribution; (ii) designing problem-specific cost matrices to describe the costs for misclassifying any particular data example; (iii) using boosting methods. Here, several methods for data augmentation were tested on the training data of the minority class only, e.g., oversampling by repeating, adding Gaussian white noises to the input data, generating synthetic minority samples using Time GAN and mixup. The performance of these preprocessing techniques in terms of four classification metrics, e.g., sensitivity, positive predictive value, specificity and negative predictive value was compared.

### Model Validation

The model performance for two different tasks, (e.g., predicting the occurrence of hypoglycemia or not and predicting both the occurrence of hypoglycemia (BG<70 mg/dL, level 1) and hyperglycemia (BG>180 mg/dL)) events is reported. Therefore, the outcome variables will be based on: 1) whether or not hypoglycemia occurred, and 2) whether or not hypoglycemia or hyperglycemia occurred. The performance of this model is measured in terms of the prediction accuracy as well as the area under the receiver operating characteristic curve (AUC). Statistical significance was tested using ANOVA.

### Example 2

To predict the risk of hypoglycemia vs. normoglycemia or hypoglycemia vs. normo-vs. hyperglycemia over different prediction time periods, neural network based transfer learning algorithms were utilized to analyze data from patients with type 2 diabetes.

### Research Design and Methods

Blood glucose values (BG), were used and measured by continuous glucose monitoring (CGM, around 5 days of data per patient were available on average and only seven consecutive BG were used in the model), to detect the occurrence of hypoglycemic or both hypoglycemic and hyperglycemic events for several time periods/prediction horizons. Patient demographics, administered medications, and laboratory results were also examined as additional variables that could potentially improve the predictive models. Three neural network models were compared (e.g., recurrent neural networks (RNNs), convolutional neural networks (CNNs) and self-attention networks (SANs)), for their ability to detect hypoglycemic events only, as well as both hypoglycemic and hyperglycemic events jointly over different prediction horizons. Blood glucose prediction results were further compared across diabetes subgroups defined by diabetes treatment (no medications vs oral medications vs. insulin use). Prediction accuracy data over different prediction horizons were compared using the area under the receiver operating characteristic curve (AUC).

### Results

Data obtained from 40 patients with type 2 diabetes was analyzed. Results indicate that the prediction accuracy of the present neural network models decrease when the prediction horizon increases for both tasks whereas the models achieve a nearly perfect prediction accuracy in short term hypoglycemia detection with little changes in the accuracy over 60 minutes. The neural network model with the best performance was the CNN model (AUC 0.99 for hypoglycemia detection and 0.96 for hypoglycemia and hyperglycemia detection, when considering a prediction horizon of 30 minutes).

### Conclusions

Excellent prediction of short term hypoglycemia has important clinical implications in terms of preventing and avoiding this potentially lethal complication, e.g., through alerts generated directly to the patient or by linking the detection algorithm to programmable insulin pumps.

### Example 3

Type 2 Diabetes is considered an epidemic worldwide. Hyperglycemia selectively damages cells that are not able to reduce glucose transport into the cell, such as capillary endothelial cells in the retina, mesangial cells in the renal glomerulus, and neurons and Schwann cells in peripheral nerves. High intracellular glucose concentration leads to the exhaustion of antioxidant pathways, altered regulation of gene transcription and increased expression of pro-inflammatory molecules resulting in cellular dysfunction and death. On a clinical level, these cellular changes translate into micro and macrovascular complications of diabetes associated with poor outcomes and increased mortality. Current diabetes treatment regimens may decrease the occurrence of complications associated with hyperglycemia, however, they also suppose a risk of extremely low glucose levels. Hypoglycemia can lead to permanent neurological damage if not treated promptly and increased mortality. The prediction of blood glucose variations helps to adjust acute therapeutic measures and food intake in patients with type 2 diabetes. Therefore, predictive algorithms that are accurate and easy to implement may facilitate better glycemic control, decrease the occurrence of hypoglycemic episodes and increase the quality of life in this population. Of note, due to the complexity of the blood glucose dynamics, the design of physiological models that produce an accurate prediction in every circumstance, (e.g., hypo/eu/hyperglycemic events), is met with restrictions.

Recently, machine learning has been shown to be very effective in solving classification and regression problems, and the ever-growing availability of already collected personal data makes the prediction of diabetic blood glucose through data-driven approaches possible.

Machine learning based data-driven approaches use the individual's recorded data, and require little understanding of the underlying physiological mechanism. Blood glucose dynamics in patients with type 2 diabetes are affected by factors such as pancreatic function, insulin levels, carbohydrate intake, history of dysglycemia and the level and extent of physical activity. Models using combinations of input parameters accounting for these factors have been considered. Many different machine learning algorithms have also been tested, including traditional machine learning algorithms (e.g., auto-regression with exogenous input (ARX), support vector machines (SVM), and Gaussian process (GP)), as well as deep learning approaches (e.g., feed-forward neural networks (FNNs), recurrent neural networks (RNNs), and convolutional neural networks (CNNs)).

Due to the remarkable effectiveness in solving classification and regression problems, deep learning has quickly become even more successful in blood glucose prediction since 2018. Among different deep learning approaches, RNNs based on the long short-term memory (LSTM), have been designed for sequence prediction problems and are the most commonly used models. However, there is no significant advantage observed by using the vanilla LSTM or convolution networks compared to a classic model (e.g., ARX), and in some cases RNNs or CNNs could showcase lower performance, as shown in a recent comprehensive benchmarking study. To achieve better prediction accuracy, more advanced network architectures have recently been developed, e.g., the recurrent convolutional neural network, which includes a multi-layer CNN followed by a RNN, and GluNet based on the Wavenet architecture. Deep learning usually requires a large amount of data to train the networks, therefore, they are usually trained by population level rather than individual level data. However, this approach cannot capture the variability of blood glucose dynamics among different patients. To address the problem of small dataset, transfer learning can be employed, which stores knowledge gained while solving one problem (e.g., population data) and then applying it to a different but related problem (e.g., patient-specific data). Transfer learning has been employed in blood glucose prediction very recently, but in these studies the patient-specific model based on transfer learning performed similarly to the population-based model or other classic machine learning models. The predictions of hypoglycemia and hyperglycemia obtained from RNN, CNN and SAN models with transfer learning techniques were compared in the setting of individual-based training. Herein, a model capable of predicting blood glucose variability in patients with type 2 diabetes with high sensitivity and specificity for the longest prediction horizon possible is proposed.

### Research Designs and Methods

Deep learning algorithms were developed for patient-specific blood glucose level prediction. Three different neural network architectures were considered, including recurrent neural networks, self-attention networks, and convolutional neural networks, and four different transfer learning strategies. Also implemented were logistic regression, Gaussian process (GP), fully-connected feedforward neural networks (FNN), and support vector machines (SVM) as the baseline models.

### Dataset

Data for this study was adopted from the cerebromicrovascular disease in elderly with diabetes study, a prospective study aimed to determine the impact of type 2 diabetes on brain tissue damage and its consequences for cognition and balance in older adults. It was conducted by Syncope and Falls in the Elderly Laboratory at Beth Israel Deaconess Medical Center (BIDMC) and approved by the institutional review board (IRB) at BIDMC. Interstitial BG recordings were measured every 5 minutes by the continuous glucose monitor (CGM). The characteristics of the patients in this study are summarized in FIG. 13. To facilitate the network training, the BG levels are scaled by 0.01 (20), and a smoothing step was applied on the BG measurements to remove any large spikes, which may be caused by patient movement.

### Predictors and Outcome

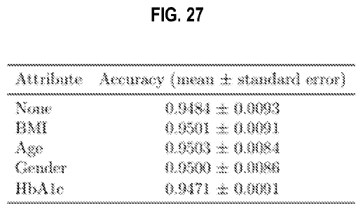

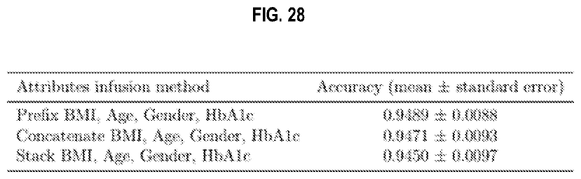

The outcome of interest in this study is the prediction of future BG values, e.g., 5, 10, 15, 20, 25, 30, 45, 60 min later. Each set of BG measured within 30 minutes (after 7 BG values) was taken as one input data segment and predict the future BG level. Additional predictors patients' characteristics like BMI, age, gender and Hb1Ac were incorporated by prefixing the scaled characteristic values in the beginning of their BG sequence; the prediction accuracy of those tests can be found in FIG. 27. Except for prefixing the scaled characteristic values, other approaches to incorporate the characteristic values and the results can be found in FIG. 28.

### Neural Network Architecture

In this study, three network architectures were employed, including recurrent neural networks (RNNs), gated convolutional neural networks (CNNs), and self-attention networks.

The typical dominant deep learning method used for sequence learning is the RNN, which is a class of neural networks that allows previous outputs to be used as the inputs of the current step. The cell units in RNNs are usually chosen as long short-term memory units (LSTM) and gated recurrent units (GRU), which deal with the vanishing gradient problem encountered by traditional RNNs. In addition to RNNs, CNNs and self-attention networks have been proposed recently for time series forecasting, and achieved better performance than RNNs in certain tasks. In gated CNNs, convolutional kernels create hierarchical representations over the input time series, in which nearby BG measurements interact at lower layers while distant BG measurements interact at higher layers. The mechanism of attention was first proposed for machine translation, and it has been shown that the network architecture based solely on self-attention mechanism can also be used successfully to compute a representation of the sequence. Self-attention is an attention mechanism to compute a representation of the sequence by relating different positions of a sequence. In RNNs, the input sequence is fed into the network sequentially, while in CNNs and self-attention networks, the input sequence is fed into the network simultaneously, and thus an embedding of the position of input elements is required. For the hyperparameters in the networks, e.g., depth and width, a grid search was performed to obtain an optimal set of hyperparameters; details of the network architectures used in this study can be found in FIGS. 7B-10B and Table 1.

### Transfer Learning Methods

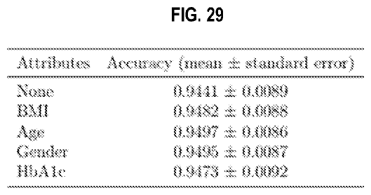

To address the difficulty of obtaining a sufficient large dataset for each patient, transfer learning was implemented on the three aforementioned neural network architectures. In transfer learning, the leave-one-out method was implemented, while the training procedure of neural networks includes two steps: first, the neural networks are pre-trained (e.g., one patient is taken out of the group as the target patient and the neural networks are pretrained on the rest of the unchosen patients). Afterwards, the target patient's data was split into two parts, the training dataset and the testing dataset. Second, the network is further retrained on the training dataset of the target patient by adopting four different approaches of transfer learning. Third, the process is repeated for other patients in the group one-by-one and obtain the prediction accuracy for each of the patients in the group on their own testing dataset. In the aforementioned pretraining step, the effect by augmenting the cohort data with an open dataset is also tested, namely the Diabetes Research in Children Network (DirecNet), which monitors the BG level in children and adolescents with type 1 diabetes, and the result is shown in FIG. 29. Two commonly-used retraining approaches are based on initialization and feature extraction. In the initialization approach the whole network is further trained, while in the feature extraction approach the last few fully-connected layers are trained from a random initialization, and other layers remain unchanged. In this study, in addition to these two approaches, a third approach is considered by combining these two approaches, (e.g., the last few fully-connected layers are further trained, and other layers remain unchanged). Details of these four transfer learning methods can be found in FIGS. 7B-10B and Table 1.

### Model Validation

The model performance is reported for two different tasks, (e.g., predicting the occurrence of hypoglycemia or not and predicting both the occurrence of hypoglycemia (BG<70 mg/dL, level 1) and hyperglycemia (BG>180 mg/dL) events). Therefore, the outcome variables were based on: 1) whether or not hypoglycemia occurred, and 2) whether or not hypoglycemia or hyperglycemia occurred. The performance of this model is measured in terms of the prediction accuracy as well as the area under the receiver operating characteristic curve (AUC). Statistical significance was tested using ANOVA.

### Sensitivity Analysis on Prediction Horizon and Subgrouping

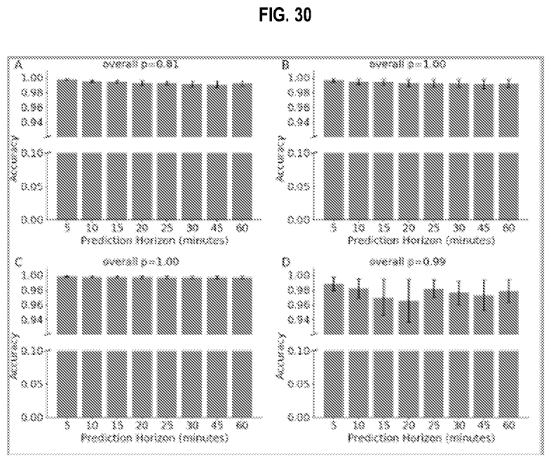

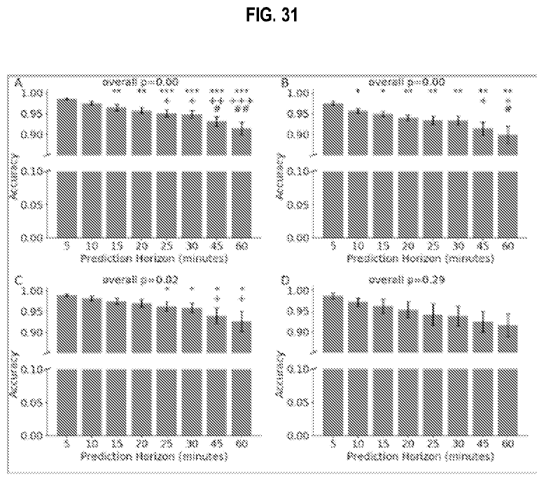

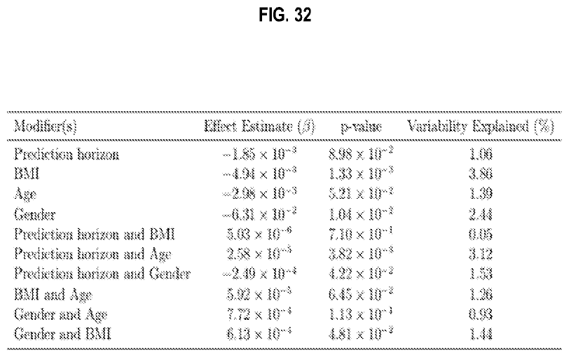

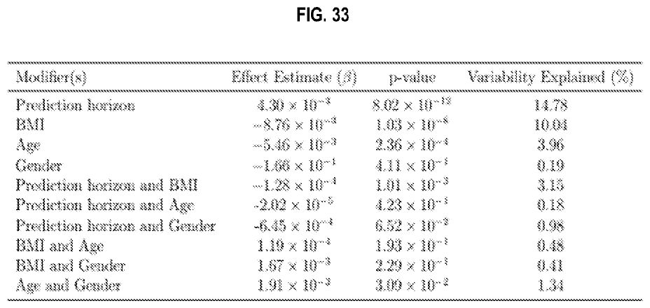

The effect of different prediction horizons on the prediction accuracy was tested by training the present models on data of the whole cohort, denoted by group “All”, or from its subgroup. Data was categorized by groups of diabetes treatment in non-treated (“Nomed”), oral hypoglycemic agents (“Oralmed”), and on Insulin (“Insulin”). FIG. 32 and FIG. 33 display the detection accuracy by diabetes regimen group for hypoglycemia and both hypoglycemia and hyperglycemia, respectively. The prediction accuracy, generally, decreases consistently when the prediction horizon becomes larger, except for hypoglycemia detection in the Insulin group. When computing the statistical significance of prediction accuracy over different prediction horizons, two vectors containing model prediction accuracy for each patient within that group were compared, where these two vectors consist of prediction accuracy for two different prediction horizons. It is suggested that the statistical differences of accuracy given different prediction horizons are not significant overall for hypoglycemia detection in FIG. 30, probably due to high prediction accuracy ranging from 95% to 99%. To further reveal the underlying interactions, effect modification was performed using ANOVA for the group “All” as an example, taking prediction horizon, BMI, age and gender along with some combinations from them as the modifiers (FIG. 32). For hypoglycemia and hyperglycemia detection, the statistical differences between different prediction horizons were quantified using ANOVA, and those prediction horizons were marked where the statistical significance becomes important (FIG. 31). Additionally, for this task, the effect modification within group “All” was tested, taking the prediction horizon, BMI, age and gender along with some combinations from them as the modifiers (FIG. 33).

### Results

### Baseline Characteristics

Data obtained from 40 patients with diabetes (19 males, mean [SD], age 65 [8] years) was analyzed. All level 1 hypoglycemic and hyperglycemic episodes from the CGM recordings were identified. A selection of the baseline characteristics of the patient cohort is reported in FIG. 13.

### Model Performance

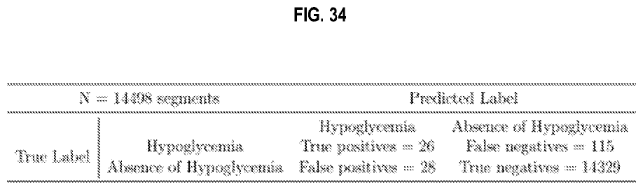

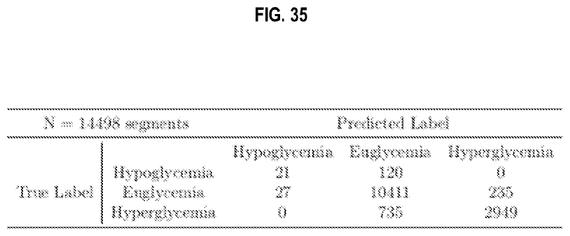

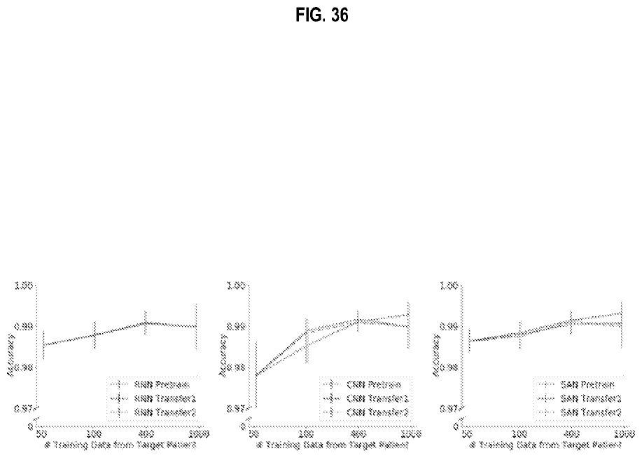

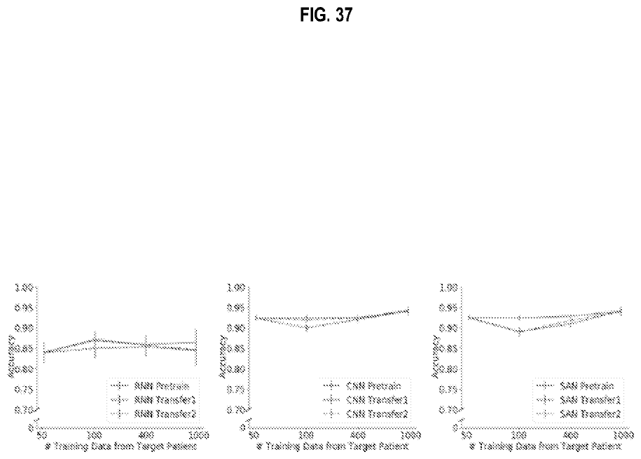

The performance metrics of the best models tested on the whole cohort, given the training data size from the target patient around 1000 data segments, are presented in FIG. 31. The AUC varied between 0.90 and 0.99 for hypoglycemia detection (binary classification, e.g., hypoglycemia vs. no hypoglycemia) and between 0.86 and 0.99 for hypoglycemia and hyperglycemia detection (three-classes classification, the output label of which is one out of these three labels, hypoglycemia, euglycemia and hyperglycemia for any given prediction). The estimation performance was better for hypoglycemia detection since there were only two possible classes/labels in a binary classification setup compared to three possible classes/labels in hypoglycemia and hyperglycemia detection. There was also a consistent decrease in AUC when the prediction horizon increases from 5 minutes to 60 minutes. FIG. 34 and FIG. 35 show the confusion matrix of the best model among the proposed models for hypoglycemia detection and hypoglycemia and hyperglycemia detection, respectively, given a prediction horizon at 30 minutes. The effect of the number of training data segments from the target patient on the prediction accuracy for these two tasks was tested; see FIG. 36 for hypoglycemia detection. FIG. 36 shows Hypoglycemia detection performance by prediction accuracy (mean and standard error) comparison among different architectures (RNN, CNN and SAN) with varying numbers of training data from the target patient, for prediction horizon (time period after the first 30 minutes of BG collection) at 30 minutes, with models trained based on the cohort (patients participated in this study) data. The data from a target patient is divided into two parts, one is for training and the other is for testing/validation. The transfer learning consists of two-step training. Step 1, train the models with the data from all the patients except for this target patient; step 2, retrain the models obtained from step 1 with the training data from this target patient. Hence, the training data size (number of training data) from the target patient is an important factor in controlling prediction accuracy. Here, the performance for all three networks is comparable. See FIG. 37 for hypoglycemia and hyperglycemia detection which shows Hypoglycemia and hyperglycemia detection performance comparison among different architectures (RNN, CNN and SAN) with varying numbers of training data from the target patient, for prediction horizon (time period after the first 30 minutes of BG collection) at 30 minutes, with models trained based on the cohort (patients participated in this study) data. The data from a target patient is divided into two parts, one is for training and the other is for testing/validation. The transfer learning consists of two-step training. Step 1, train the models with the data from all the patients except for this target patient; step 2, retrain the models obtained from step 1 with the training data from this target patient. Hence, the training data size (number of training data) from the target patient is an important factor in controlling prediction accuracy. Here, CNN Pretrain and Transfer2 as well as SAN Pretrain have the best performance. FIG. 36 shows that all of the three proposed neural networks can achieve consistently better accuracy and relatively high accuracy, given more training data from the target patient in hypoglycemia detection. However, FIG. 37 shows that CNN and SAN could maintain consistently increasing accuracy when more training data from patients is given. Specifically, the inference results show that CNN slightly outperforms SAN in most of the conditions. The present models were compared with some existing classification methods, logistic regression, GP, SVM and FNN in terms of 1) Hypoglycemia detection (FIG. 16A), 2) Hypoglycemia and hyperglycemia detection (FIG. 16B) over a prediction horizon of 30 minutes, and 3) Hypoglycemia detection (FIG. 16C), 4) Hypoglycemia and hyperglycemia detection (FIG. 16D) over a prediction horizon of 60 minutes. In both tasks, the model showed consistent increase in accuracy and AUC given more training data from the target patient and, specifically, better than other models in hypoglycemia and hyperglycemia detection.

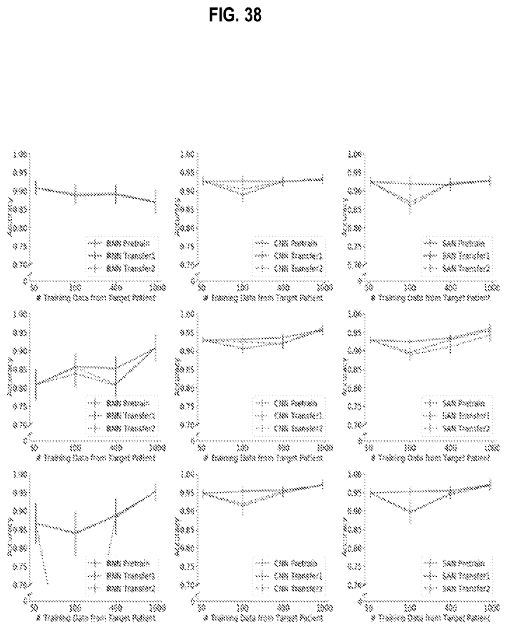

FIG. 38 shows hypoglycemia and hyperglycemia detection performance comparison among different architectures (RNN, CNN and SAN) with varying numbers of training data from the target patient, for prediction horizon (time period after the first 30 minutes of BG collection) at 30 minutes. (Top) Models are trained on the subgroup of patients taking no medication. (Middle) Models are trained on the subgroup of patients taking oral medication. (Bottom) Models are trained on the subgroup of patients taking insulin. The data from a target patient is divided into two parts, one is for training and the other is for testing/validation. The transfer learning consists of two-step training. Step 1, train the models with the data from all the patients except for this target patient; step 2, retrain the models obtained from step 1 with the training data from this target patient. Hence, the training data size (number of training data) from the target patient is an important factor in controlling prediction accuracy. Here, for all subgroups, CNN Pretrain and Transfer2 have the best performance.

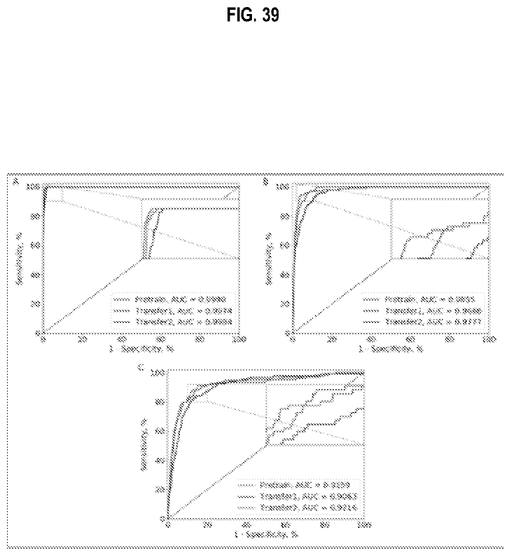

FIG. 39 shows hypoglycemia detection (binary classification, e.g., hypoglycemia vs. no hypoglycemia) ROC curves and the corresponding area under curve (AUC) of the best model (CNN) for the following prediction horizons. (A) 5 minutes, (B) 30 minutes, and (C) 60 minutes. Pretrain refers to the transfer learning method where the model is trained on the data from all patients except for the target patient, retrain the model on the training data from the target patient by keeping the model parameters fixed and then test the model on the testing data from the target patient. Transfer1 and Transfer2 follow a similar procedure as Pretrain does except for the retraining step, where the model parameters are allowed to change in two different manners. A third transfer learning method was also tested, namely Transfer3, which follows a similar procedure as Pretrain does except for the retraining step, where the model parameters are allowed to change in a different manner compared to Transfer1 and Transfer2. However, the results by Transfer3 are not as good as Pretrain, Transfer1 and Transfer2, hence is omitted here.

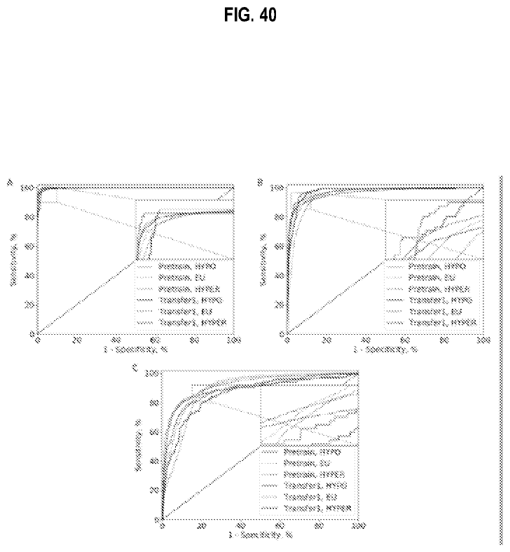

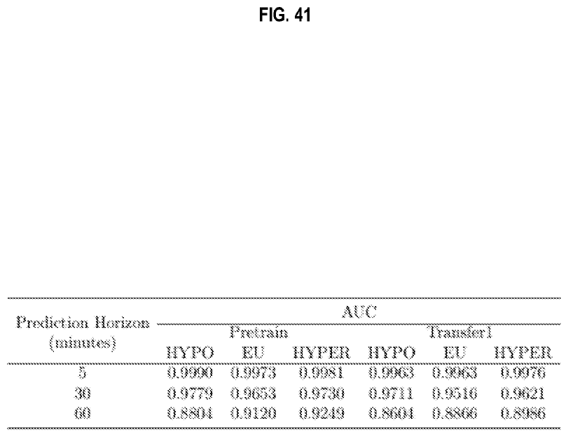

FIG. 40 shows hypoglycemia and hyperglycemia detection (three-class classification) ROC curves and the corresponding area under curve (AUC) of the best model (CNN) for the following prediction horizons. (A) 5 minutes, (B) 30 minutes, and (C) 60 minutes. In three-class classification, the output label of prediction is one out of three labels, “hypoglycemia”, “euglycemia” and “hyperglycemia”, at any given time. Note that in this task, the labels (HYPO for label “hypoglycemia”, EU for label “euglycemia” and HYPER for label “hyperglycemia”) were iterated over. For example, one of the labels, “hypoglycemia”, was set as the TRUE label and treated the rest of the labels, e.g., “euglycemia” and “hyperglycemia”, together as the FALSE label to generate the ROC curves. Detailed AUC values for the ROC curves presented are summarized in FIG. 41. Pretrain refers to the transfer learning method where the model is trained on the data from all patients except for the target patient, retrain the model on the training data from the target patient by keeping the model parameters fixed and then test the model on the testing data from the target patient. Transfer1, Transfer2 and Transfer3 follow a similar procedure as Pretrain does except for the retraining step, where the model parameters are allowed to change in three different manners. Since the results by Transfer2 are comparable to those by Pretrain while those by Transfer3 are not as good as Pretrain, Transfer1 and Transfer2, while only the results of Pretrain and Transfer1 are shown for brevity.

FIG. 39 shows hypoglycemia and hyperglycemia detection area under ROC curves (AUC) summary. The AUC scores are summarized from FIG. 38. Note that in this task, the labels (HYPO for label “hypoglycemia”, EU for label “euglycemia” and HYPER for label “hyperglycemia”) were iterated over. For example, one of the labels, “hypoglycemia”, was set as the TRUE label and treated the rest of the labels, e.g., “euglycemia” and “hyperglycemia”, together as the FALSE label to generate the ROC curves. Prediction horizon, time period after the first 30 minutes of BG collection.

### Conclusion

In this study, deep learning algorithms were utilized for hypoglycemia and hyperglycemia detection for patients with Type 2 diabetes. Three neural network models were considered, including recurrent neural networks (RNNs), convolutional neural networks (CNNs) and self-attention networks (SANs). Four transfer learning strategies were examined, which enable training the neural networks with a small amount of individual's recorded data. The performance of the algorithms on the data obtained from 40 patients was demonstrated.

High prediction accuracy was achieved for the detection of hypoglycemic events with accuracy no less than 98% and AUC greater than 0.9 for all the prediction horizons examined when the model was trained on the dataset of all patients in the cohort, namely group All. For the detection of both hypoglycemia and hyperglycemic events, the best model among all tested models achieved high accuracy greater than 89% and AUC greater than 0.86, for all the prediction horizons examined. The results indicate that as the prediction horizon prolongs, the prediction accuracy as well as AUC decrease in both classification tasks.

Model training by diabetes treatment was incorporated into a not-treated with medications (“Nomed”) group, an oral hypoglycemic agents (“Oralmed”) group, and an on Insulin (“Insulin”) group, over several prediction horizons for hypoglycemia and both hyperglycemia and hypoglycemic events. The results show that the overall prediction accuracy is the highest in the “Oralmed” group for both prediction tasks. Patients with Type 2 Diabetes are often on multiple oral hypoglycemic agents, which have extended half-lives, or combination of oral agents with insulin. The extended action of such medications challenges the adjustment for provoked small variations in blood glucose during the day. Therefore, the findings make the use of prediction algorithms more applicable in this group of patients. When comparing the model performance on predicting hypoglycemia and both hyperglycemia and hypoglycemic events, it was determined that for the same group where the model was trained on, the overall prediction accuracy and AUC in hypoglycemia only detection may be higher than those in hypoglycemia and hyperglycemia detection.

The results showed that statistical significance was observed in the hypoglycemia and hyperglycemia detection task when models were trained on group “All”, group “Nomed” and group “Oralmed”, but not in any group for the hypoglycemia only detection task. More specifically, statistical significance was observed between two short prediction horizons (5 min and 10 min) and the largest prediction horizon (60 min) in the hypoglycemia and hyperglycemia detection, when models were trained on group “All”, group “Nomed” and group “Oralmed.” It is noted that despite statistical differences observed among different prediction horizons, the model always maintained high accuracy. Two-way ANOVA was applied to investigate the effect of other demographic attributes of patients besides the prediction horizon, like BMI, age, gender and the corresponding second-order interaction terms between pairs of these attributes. The results showed that the prediction accuracy of hypoglycemia and both hyperglycemia and hypoglycemia prediction is affected by the patient's demographics, which indicates that these two tasks are innately different. This is clinically understandable as for instance, obesity or elderly age pose higher glucose variability and make it difficult to obtain glycemic control.

To summarize, a new method was proposed for predicting hypoglycemia and/or hypoglycemia and hyperglycemia detection showing remarkable performance characterized by high prediction accuracy and AUC. Time periods up to 60 minutes were the focus in this work because it is believed that accurate hypoglycemia prediction over this period of time offers the most in terms of having potential warning signs and preventing adverse events by hypoglycemia. By incorporating transfer learning, this method could provide patient-specific hypoglycemia and hyperglycemia detection with just a few blood glucose samples from the targeted patient. Despite the high accuracy and a few training data demanded by the present method, there are some limitations to current work. Different from other physiologically-derived algorithms, this method is purely data-driven with no physiological knowledge, and performs prediction merely based on the blood glucose history. It is recognized that data-driven methods are double-edged swords. On one side, data-driven methods relieve physicians from exhausting all possible combinations of physiological inputs given large samples or data. On the other side, it is not an easy task to incorporate domain knowledge to data-driven methods, especially in neural network based models. In the present study, nutritional intake, exercise or stress conditions in dysglycemia detection were identified as the domain knowledge, the appropriate incorporation of which could possibly improve the model accuracy. Hence, it is proposed to develop physiologics-informed neural network models in future work. This and similar algorithms in the future are expected to have important clinical implications in terms of preventing and avoiding this potentially lethal complication (e.g. through alerts generated directly to the patient or by linking the detection algorithm to programmable insulin pumps).

### Example 4

### Methods

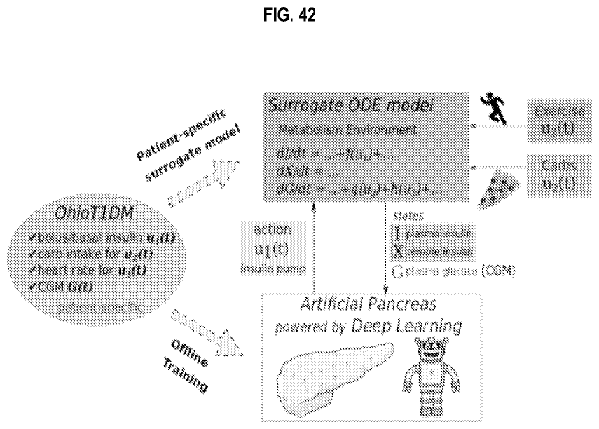

Problem Setup: A framework to design patient-specific automated insulin delivery system for six patients with type 1 diabetes using patient-specific metabolic data, namely the OhioT1DM dataset, was developed. The OhioT1DM dataset contains eight weeks' worth of continuous glucose monitoring, insulin, physiological sensor, and self-reported life-event data for each of 12 people with type 1 diabetes, in which 6 patients appear in the 2018 version and the other 6 patients in the 2020 version of the dataset. A workflow of this work is shown in FIG. 42. FIG. 42 shows exogenous insulin (bolus insulin+basal insulin, u1(t)), carbohydrate intake, u2(t); heart rate, u3(t); and CGM measured glucose level G(t). On the one hand, the OhioT1DM dataset is used for parameter inference of the patient-specific ODE model, providing required records of total exogenous insulin (bolus insulin+basal insulin), carbohydrate intakes, heart rate, CGM measured glucose level. On the other hand, to build the automated insulin delivery system, we use the OhioT1DM dataset to train the offline reinforcement learning algorithm. The final optimized agent powered by the deep neural networks will serve as the patient-specific artificial pancreas.

OhioT1DM dataset: All data contributors in the OhioT1DM were on insulin pump therapy with continuous glucose monitoring (CGM), throughout the 8-week data collection period. In these 8 weeks of monitoring, the patients also reported life-event data via a custom smart-phone app and provided physiological data from a fitness band. Based on the Roy model, we select a subset of the data including 1) the CGM blood glucose level measure every 5 minutes, 2) insulin doses, both bolus insulin and basal insulin, 3) self-reported meal times with carbohydrate estimates and 4) the heart rate measured every 5 min. We note here that only the data of those 6 patients in the 2018 version is used in our analysis, given the data of patients in 2020 version does not include heart rate monitoring.

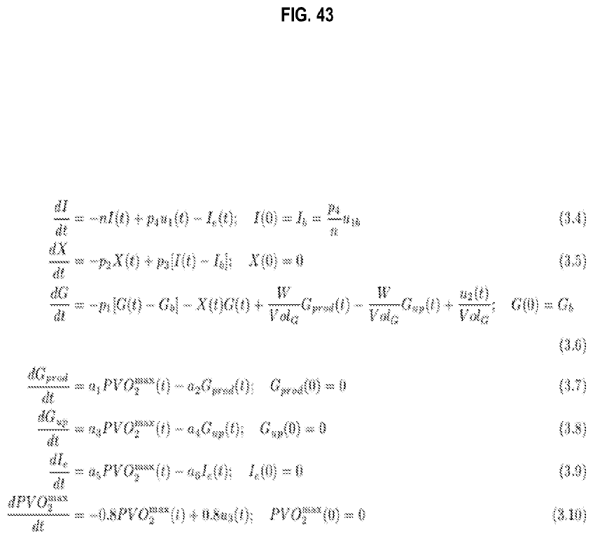

We adopt a slightly simplified version of the ODE system by omitting the _{Ggly}(t) term representing the decline of the glycegenolysis rate during prolonged exercise due to the depletion of liver glycogen stores, since the patients we consider only perform sporadic and light physical activities. The resulting ODE system for the 7 state variables X1, . . . , 7=[I, X G, Gprod, Gup, Ie, PVO2max], shown in Eqs. (3.4)-(3.10), in FIG. 43, captures the exercise-induced dynamics of plasma insulin clearance and the elevation of glucose uptake and hepatic glucose production rates.

Data Preprocessing: The exogenous insulin is calculated as the sum of basal insulin and bolus insulin at each minute. According to the user manual of the insulin pumps, i.e., Medtronic 530G and 630G, while the basal insulin is given at a rate and is provided explicitly, bolus insulin is a one-time dose and can be released into the blood stream using different modes, i.e., “normal”, “normal dual”, “square” and “square dual”. Given limited information for the exact releasing process of these different modes in the OhioT1DM dataset, we manually define the conversions through literature and the user guides for the insulin pump. In “normal” type, the single x mU dose bolus insulin is converted to a constant pseudo basal insulin at a rate of x/10 mU lasting for 10 minutes. In “square” type, the single x mU dose bolus insulin is converted to a constant pseudo basal insulin at a rate of x/t mU lasting for t minutes, where t denotes the time elapse between two adjacent boluses. In “normal dual” and “square dual”, the single dose is divided evenly into two identical half doses. These two half doses are released sequentially by “normal” and “square” type in “normal dual”, while by “square” and “normal” type in “square dual”, respectively. Additionally, we also consider “temp basal” insulin to override the basal insulin set previous. The glucose insulin rate is computed by the meal carb intakes using an exponential decay function as follows,

$$N$$

$$IG ( t ) = 0.0083 \times mj exp ( 0.0083 ( tj - t ) ) ,$$

$$j = 1$$

where mj gram of carb intake is recorded at tj. The percentage of VO²max (PVO2max) denoting the exercise intensity is approximated by the heart rate as follows:

$${PVO}_{2}^{max} ( t ) = \text{.888} HR - 71.91 ,$$

$${u}_{3} ( t ) = {PVO}_{2}^{max} ( t ) - 8 , if {u}_{3} ( t ) > 0 else 0$$

where 8 denotes basal VO₂^{max} at 8%, and HR denotes heart rate.

System Biology Informed Neural Networks (SBINNs): A new systems-biology-informed deep learning framework, namely system biology informed neural networks (SBINNs), was developed which successfully incorporates the system of ordinary differential equations (ODE) into the neural networks. Inspired by the physics-informed neural networks, SBINNs is sequentially composed of a input-scaling layer to allow input normalization for the robust performance of the neural networks, a feature layer marking different patterns of state variables in the ODE system and the output-scaling layer to convert normalized state variables back to physical units. By effectively adding constraints derived from the ODE system to the optimization procedure, SBINNs is able to simultaneously infer the dynamics of unobserved species, external forcing, and the unknown model parameters.

Given the measurements of y₁, y₂, . . . y_{M} at the time t₁, t₂, . . . , t_{N}data, SBINNs enforce the network to satisfy the ODE system at the time point τ₁, τ₂, . . . , τ_{N}^{node}. The total loss is defined as a function of both the parameters of the neural networks, denoted by θ and parameters of the ODE system, denoted by ρ.

$$- ℒ ( 0 , p ) = - {ℒ}^{data} ( 0 ) + - {ℒ}^{ode} ( 0 , p ) + - {ℒ}^{aux} ( 0 )$$

where £^{data} is associated with the M sets of observations of the state variables y in the ODE system; £^{ode} enforces the structure imposed by the system of ODEs; £^{aux} is introduced as an additional source of information for the system identification. The final step of SBINNs is to infer the neural network parameters θ as well as the unknown ODE parameters ρ simultaneously by minimizing the aforementioned loss function via gradient-based optimizers. The known observed state variable y is the CGM measured glucose record in the OhioT1DM dataset, i.e., G(t), which is used for minimizing the data loss, £^{data}. We use £^{ode} to minimize the residue of ODE system, shown in Eqs. 3.4-3.10 in FIG. 43.

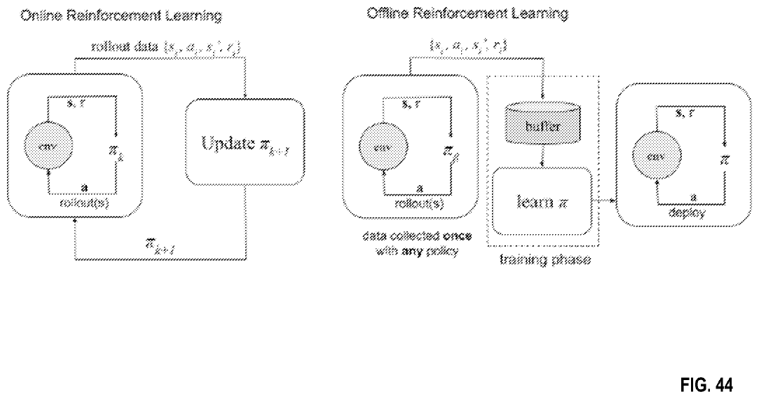

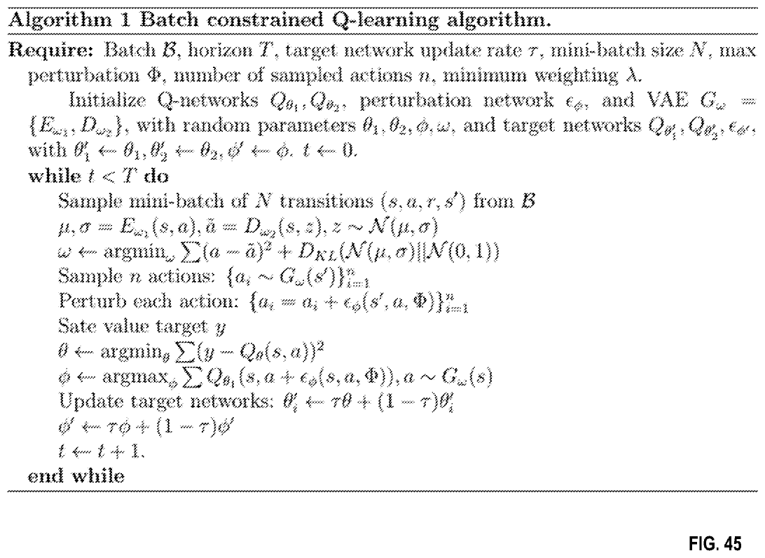

Offline Reinforcement Learning: While (online) reinforcement learning is the problem faced by an agent that must learn behavior through trial-and-error interactions with a dynamic environment, offline reinforcement learning algorithms utilize previously collected data, without additional online data collection through interacting with the environment. As shown in FIG. 44, in online reinforcement learning, the policy Rx is updated with streaming data collected by πκ. In offline reinforcement learning, a buffer dataset is collected by some behavior policy πβ. The dataset is collected once, and is not altered during training. Finally, the policy is only deployed after being fully trained. In this work, we generate a buffer by sampling the states like glucose level, carb intakes and exercise, the action denoted by total exogenous insulin, i.e., sum of bolus insulin and basal insulin, and the corresponding returns. In FIG. 44, s denotes the states of the environment; r denotes the return which is the sum of value of each state in the past. π denotes the policy which is a function of state s and action a. πκ denotes the policy at k-th iteration. πβ denotes a behavioral policy used for one-time data collection. The batch constrained Q-learning (BCQ) algorithm, which is one of the most popular online reinforcement learning algorithms, was implemented. By restricting the action space in order to force the agent towards behaving close to a subset of the given data, BCQ is able to learn successfully without interacting with the environment by considering extrapolation error. The details of BCQ algorithm is shown in Algorithm 1 of FIG. 45. We use fully-connected feed forward neural networks for the Q-networks and the variational autoencoders (VAE).

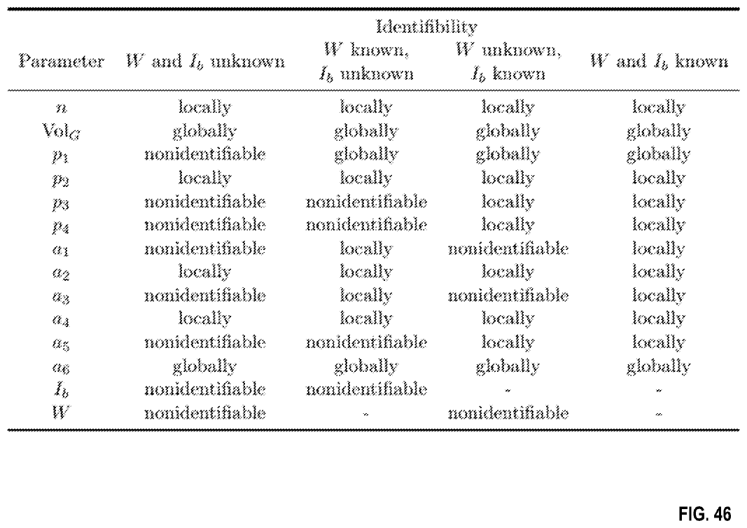

Patient Specific Model Using SBINNs: To build a surrogate environment for our agent to interact with, we perform parameter inference using system biology informed neural networks (SBINNs) on the Roy model. We first perform structural identification on the ODEs the set of parameters {n, VolG, p1, p2, p3, p4, a1, a2, a3, a4, a5, a6, W, Ib} in Eqs. (3.4)-(3.10) of FIG. 43. Despite that most of the ODE parameters are not readily available in the OhioT1DM dataset, there are several parameters that are practically available for patients in future applications. For example, patient's body weight (W) can be measured practically, but is not available in OhioT1DM. While the basal plasma insulin (Ib) is not available in the OhioT1DM, but it can be obtained using blood test. We consider the identifiability of the aforementioned parameters under different scenarios depending on whether W and Ib are known for the given patient. FIG. 46 suggests that when W and Ib are both known, other parameters are either globally identifiable or locally identifiable. This scenario was adopted in the following analysis by assuming W=60 kg, and construct patient-specific model by inferring the set of parameters {n, VolG, p1, p2, p3, p4, a1, a2, a3, a4, a5, a6, W, Ib} using patient-specific plasma glucose record in one day. In FIG. 46, W denotes the body weight of patient, Ib denotes the basal plasma insulin level.

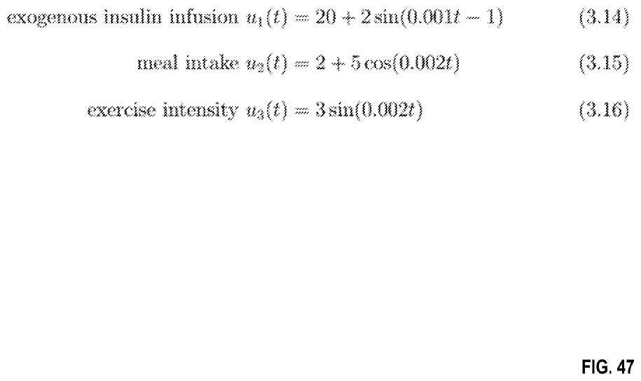

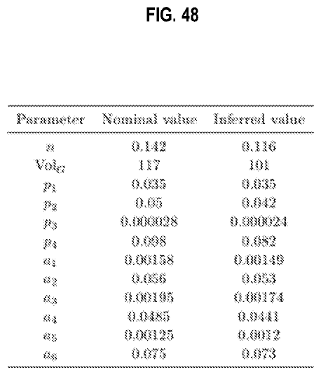

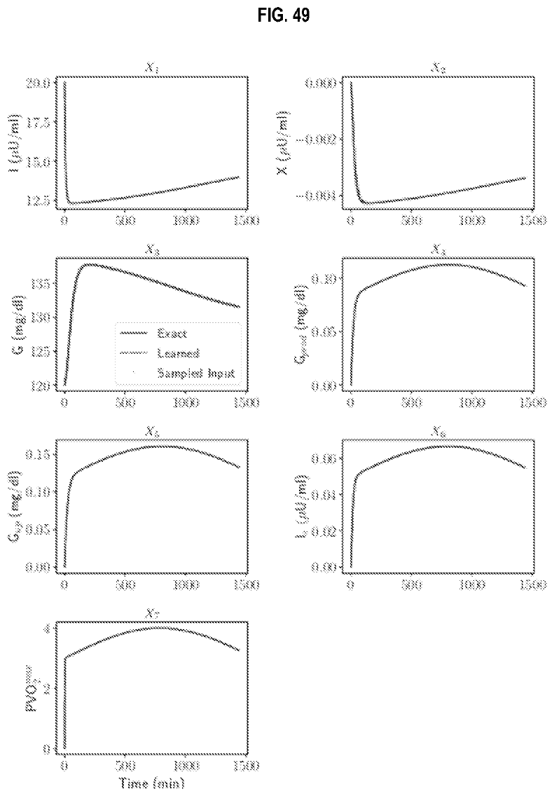

Parameter Inference Using Synthetic Data: In a toy model, it was assumed the weight of a virtual patient to be W=60 kg and synthetic data was generated by solving an initial value problem, where the forcing terms in the ODE system are given by Eqs. (3.14)-(3.16) of FIG. 47; the model parameters are given by the nominal value in FIG. 48); and the initial condition of the ODE system is given by [I, X G, G_{prod}, Gᵤₚ, PVO₂max]t=[20, 0, 120, 0, 0, 0, 0]. As required by SBINNs, we take the one-day glucose record, the only state variable available in the OhioT1DM dataset among those 7 variables in the Roy model, to be the known state variable. Hence, the data loss L_{data} denotes the mean squared error (MSE) between the known one-day glucose record and the model predicted glucose dynamics. For the auxiliary loss Lₐᵤₓ, we impose the sum of the MSE between the synthetic data and the model prediction of the state variables at the initial moment and the MSE between them at the terminal moment. FIG. 48 and FIG. 49 show the parameters and the time-dependent dynamics of the state variables, both the known state G and the other hidden states, inferred by SBINNs, respectively. These result suggest SBINNs is capable of inferring the model parameters as well as the hidden dynamics of unobserved states, i.e., [I, X Gprod, Gup, Iₑ, PVO₂^{max}] with high accuracy. FIG. 48 shows parameter inference by SBINNs for a modified model using data from a synthetic case. FIG. 49 shows prediction of the 7 state variables using SBINNs for the synthetic case. In FIG. 49, the one-day glucose record (blue points in subfigure X3=G) is given. The blue solid line denotes the numerical solution of the system of ODEs. The red dash line denotes the prediction by SBINNs. SBINNs can infer all state variables correctly both the observed state variable (X3) and hidden state variables, (X1, X2, X4, X5, X6, X7).

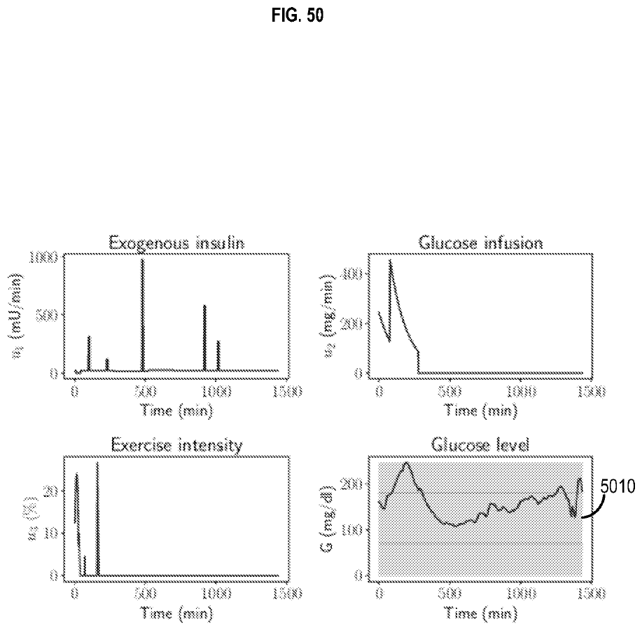

Patient-specific Parameter Inference using OhioT1DM: In the OhioT1DM dataset, we assume the patient's weight is 60 kg, i.e., W=60 kg. The initial condition of the ODE system is given by [I, X, G, Gprod, Gup, Ie, PVO₂max]t=0=[Ib, 0, Gb, 0, 0, 0, 0], where Ib is roughly estimated from the basal insulin rate and Gb is given by the CGM recording. In contrast to the toy case, we only impose the initial condition for the auxiliary loss in this real-world case. We also impose smoothing using a moving window of 30 data points on the model inputs to remove the noise and hence speed up the convergence of parameter inference. FIG. 50 shows model inputs required for the model in a real-world case. The central 5010 region denotes normoglycemia, i.e., glucose levels within 70 mg/dl and 180 mg/dl, while red region denotes either hypoglycemia or hyperglycemia, i.e., glucose levels below 70 mg/dl or above 180 mg/dl, respectively.

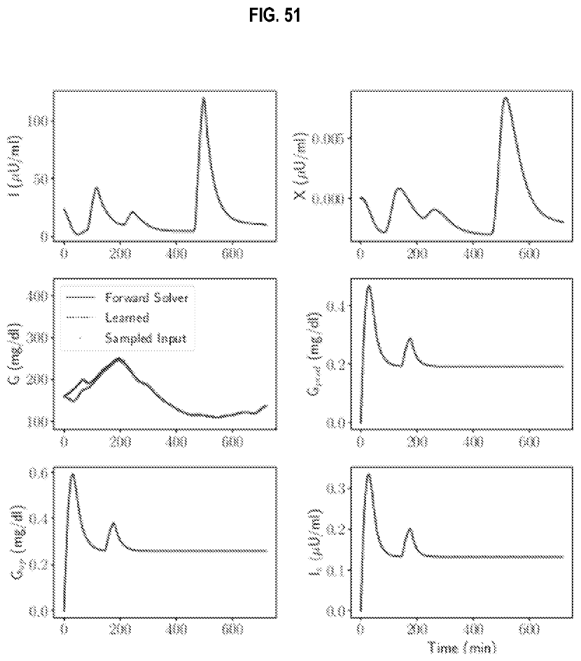

FIG. 51 shows prediction of the 7 state variables in the model using SBINNs for a real-world case. In this case, the half-day glucose record (blue points in subfigure X3=G) is given. The solid line denotes the numerical solution of the system of ODEs, using inferred parameters. The dash line denotes the prediction by SBINNs. SBINNs can infer all state variables correctly both the observed state variable (X3) and hiding state variables (X1, X2, X4, X5, X6, X7).

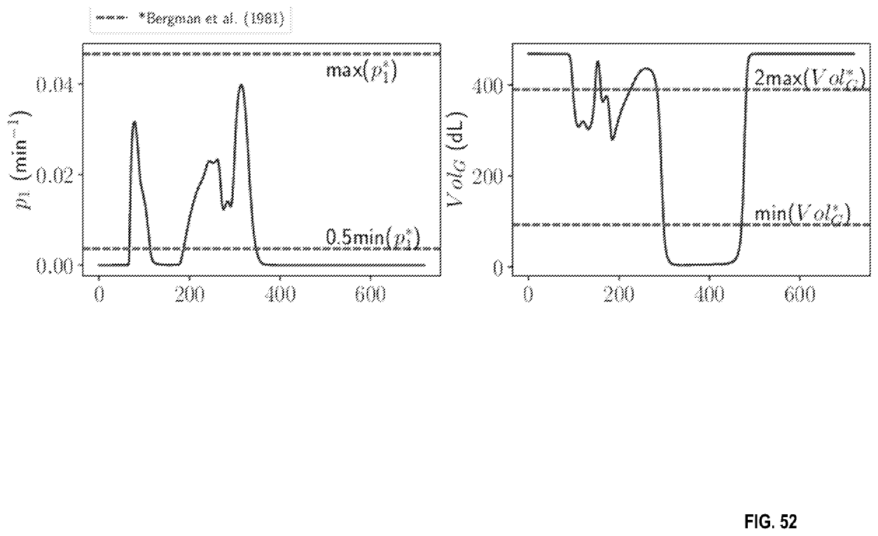

FIG. 52 shows inferred time-dependent parameters for a real-world case. In FIG. 52, the dashed lines denote the thresholds calculated from 18 human subjects.

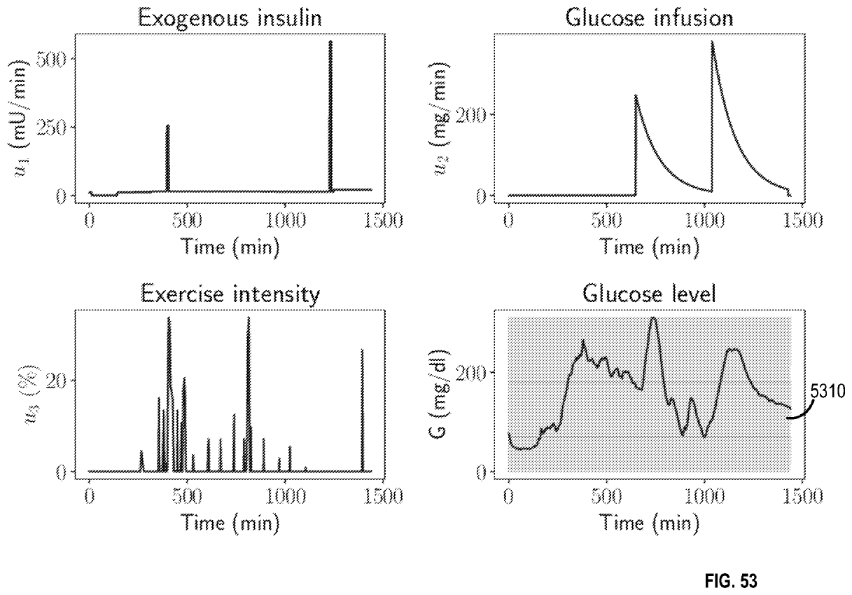

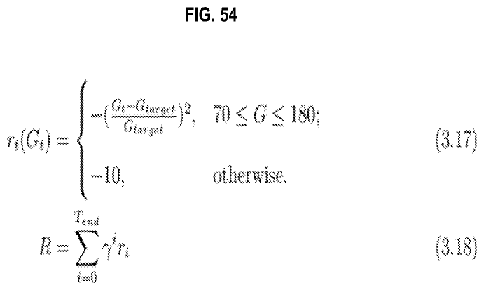

FIG. 53 shows model inputs required for the model in real-world case with violent glucose levels ranging from hypoglycemia, normoglycemia, and hyperglycemia. A central region 5310 denotes normoglycemia, i.e., glucose levels within 70 mg/dl and 180 mg/dl, while the regions above and below denote hypoglycemia and hyperglycemia respectively with glucose levels outside of 70 mg/dl to 180 mg/dl. The reinforcement learning problem for glucose regulation was formulated using the glucose infusion rate u₂ and exercise intensity u₃ from the OhioT1DM dataset, and the agent was trained to provide the optimal exogenous insulin infusion rate u1 for a full day period. The observed states sₜ at time t is the glucose levels in the past 30 minutes, i.e., sₜ=[Gₜ₋₂₉, Gₜ₋₂₉, . . . , Gₜ]. The action aₜ at time t is a value within the range [0, 740] mU/min. Informed by the prior knowledge of the danger due to hyperglycemia and hypoglycemia, we design the reward function such that the reward penalties much more on actions leading to glucose levels falling into hypoglycemia or hyperglycemia. The reward function at time t is given by Eq. 3.17 and the final return function is given by Eq. 3.18 in FIG. 54. A simplified patient-specific model was built using the parameters inferred with SBINNs, For the time dependent parameters, p1 and VolG, we use the corresponding mean values to simplify the parameterization of the simulation. In this case, the forcing terms required by the model, i.e., the exogenous insulin infusion rate u1, glucose infusion rate u2 and the exercise intensity u3 are shown in FIG. 54.

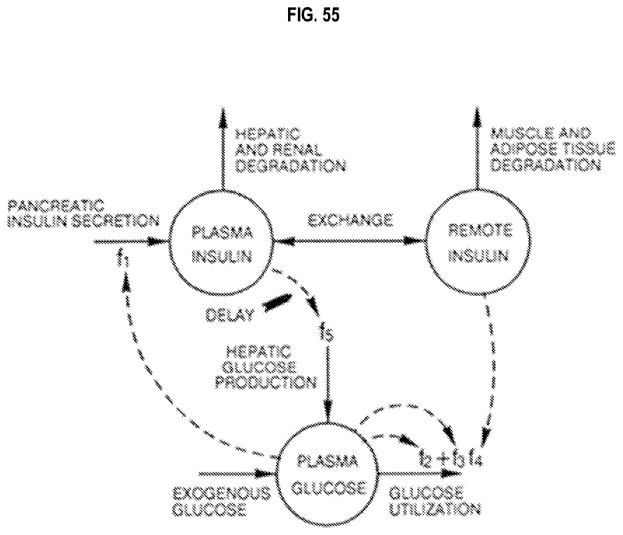

FIG. 55 shows an ultradian model. The developed algorithm has the capability to infer unknown parameters and dynamics for the glucose-insulin interaction based on the ultradian model. The model has 6 state variables and 30 parameters. In this model, the main variables are the plasma insulin concentration Ip the interstitial insulin concentration Ii, and the glucose concentration G. The nutritional driver IG is the systematic forcing of the model that represents the external sources of glucose from nutritional intake. Among these variables, only the glucose level G can be easily measured. The model is trained only with the glucose data and can (1) infer the unknown parameters in the model, and (2) infer the dynamics of plasma insulin concentration and interstitial insulin concentration, which cannot be easily observed in practice. Moreover, once we have inferred the parameters, we can then accurately forecast the glucose levels and insulin concentration in the future.

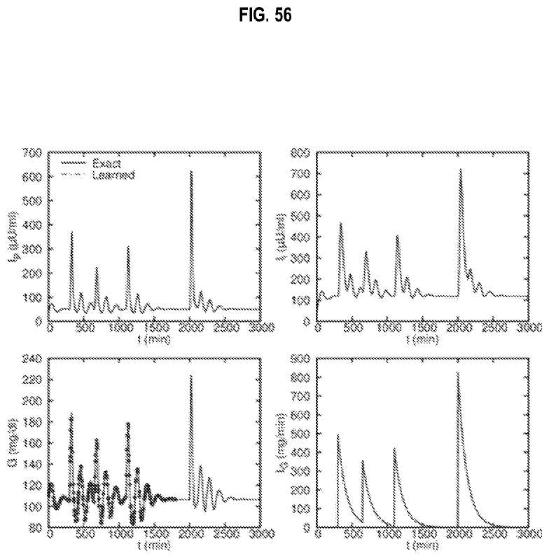

FIG. 56 shows the ultradian glucose-insulin inferred dynamics and forecasting compared with the exact solution given nutrition events. 600 scattered glucose level (data points) are measured from 0-1800 min and used for parameter identification and dynamics inference. Given the inferred parameters, we can accurately forecast the glucose levels following the event at time t=2000 min.

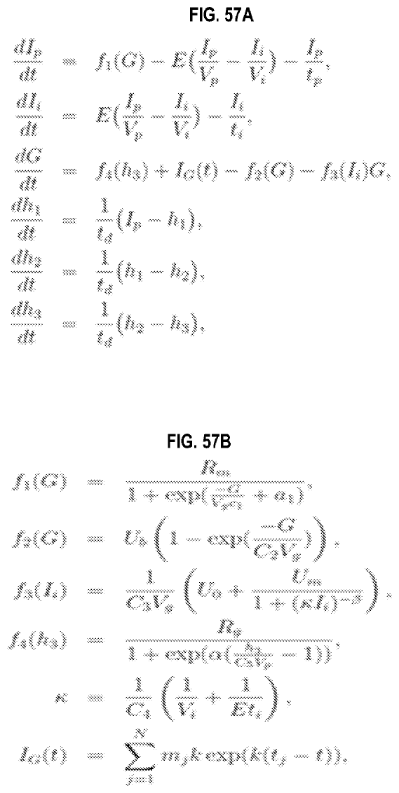

The system of equations of the glucose-insulin interaction model is given by the equations in FIG. 57A and for which the major parameters include: (i) E, a rate constant for exchange of insulin between the plasma and remote compartments; (ii) IG, the exogenous (externally driven) glucose delivery rate; (iii) tp, the time constant for plasma insulin degradation; (iv) ti, the time constant for the remote insulin degradation; (v) td, the delay time between plasma insulin and glucose production; (vi) Vp, the volume of insulin distribution in the plasma; (vii) Vi, the volume of the remote insulin compartment; (viii) Vg, the volume of the glucose space. Further, f1(G) represents the rate of insulin production; f2(G) represents insulin-independent glucose utilization; f3(Ii) represents insulin-dependent glucose utilization; f4(h3) represents delayed insulin-dependent glucose utilization. Where f1-f4 and the nutritional driver of the model IG(t) are given by the equations in FIG. 57B, where IG(t) is defined over N discrete nutrition events with k as the decay constant and event j occurs at time tj with carbohydrate quantity mj. The nutritional driver of the model is the intake IG(t) defined over N discrete nutritional events.

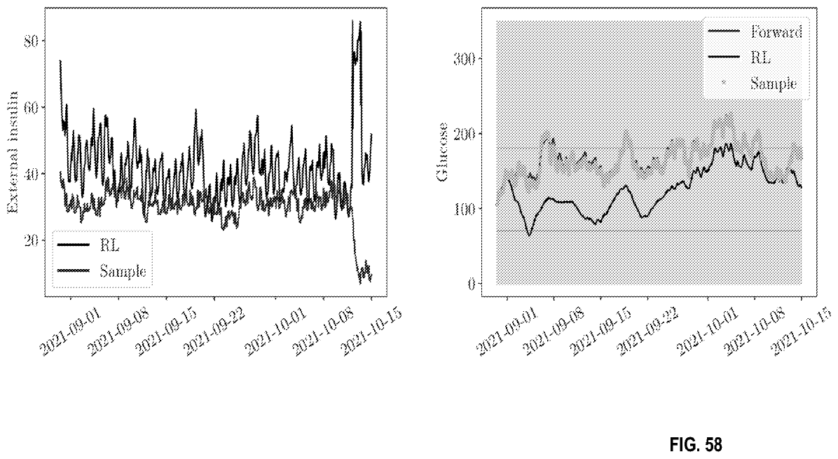

The target glucose level was set to be 120 mg/dl and the terminal time T_{end} to be 1439 min, for a total of 1440-min episode. The BCQ is capable of outperforming the patient's self-prescribed insulin dosage resulting in a baseline return −2238.14. The performance of the best agent is given by the solid curve in the right subfigure of FIG. 58. The lower curve in the left figure denotes the sampled external insulin infused/injected from the OhioT1DM dataset, resulting in the glucose trajectory (forward curve) in the right figure and the upper curve in the left figure denotes the learnt external insulin infused/injected by off-line RL, resulting in the glucose trajectory (RL curve) in the right figure.

The central region (70<glucose<180) in the right subfigure denotes clinically acceptable glucose levels, while the regions on the top and bottom denote hyperglycemia and hypoglycemia region. Note that we could adjust the acceptable glucose levels could be adjusted, for example by a caregiver, on a per patient basis, i.e., adjusting 70 and 180 lower or higher, to achieve either more strict or less strict glucose management. In the right figure, by using RL, the time curve fall into the central region (clinically defined as time-in-range) is longer than that the out of range curve does, indicating that time in range is improved by RL. The sample dots in the right figure denotes the sampled glucose from the dataset using continuous glucose monitoring (CGM).

### Conclusions

Overestimation and/or underestimation of insulin dosing can be extremely dangerous. The present methods and systems describe a novel framework with effectively combines real-world historical media data, containing patient-specific glucose, insulin, meal intake and exercise, with a flexible ODE model defining glucose-insulin dynamics by systematically prioritizing two significant external factors, i.e., meal intake and physical activities, and an offline reinforcement learning algorithm, namely batch constraint Q-learning, which is capable of developing an efficient agent with no need of interacting with a target environment. The present methods and systems describe a biology informed neural networks (SBINNs) to infer patient-specific parameters of the ODEs model using patient-specific data, i.e., glucose trajectory, glucose infusion rate, exogenous insulin infusion rate, and exercise intensity. After validated the parameters inferred by SBINNs, we train an agent to automate insulin infusion and evaluate the performance of the agent on the patient-specific ODE model. The evaluation results suggest that the best trained agent has a better performance in terms of maintaining blood glucose levels within the safe range, i.e., within 70 mg/dl and 180 mg/dl, comparing to the self-modulated insulin infusion on the patient's own.

Despite the improved glycemic control provided by our offline agent compared to that by the patient and minimal human intervention demanded by our framework, we could still identify possible improvements to the proposed framework. Although we correctly identify the patient-specific parameters required in the Roy model from one-day glucose data, we may still need to address the variability of the parameters in practice.

One possible solution to this practical problem is to adapt the constant parameters to stochastic parameters represented by neural networks. Since current model consider only patients with type 1 diabetes, i.e., the 9 cell of the patient's pancreas secret very little insulin, we must adjust the ODEs model to consider the insulin resistance, to build models for patients with type 2 diabetes. It has been suggested suggest that the occurrence of sustained insulin and glucose oscillation is dependent on 1) a time delay of 30-45 min for the effect of insulin on glucose production and 2) a sluggish effect of insulin on glucose utilization, because insulin acts from a compartment remote from plasma. To incorporate these characteristics to the numerical model, we need to modify the ODEs system accordingly to address the time delay between insulin and glucose.

While specific configurations have been described, it is not intended that the scope be limited to the particular configurations set forth, as the configurations herein are intended in all respects to be possible configurations rather than restrictive. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A method comprising:
receiving, by a predictive model, current blood glucose data from the patient, wherein the predictive model is trained on previous blood glucose data from a population and previous blood glucose data from a patient;
determining, based on the current blood glucose data, one or more future blood glucose values for the patient, wherein the predictive model; and
determining the one or more future blood glucose values satisfies a threshold; and
based on the one or more future blood glucose values satisfying a threshold, causing an administration of insulin.

2. The method of claim 1, wherein the current blood glucose data comprises one or more physiological parameters including one or more of ingested carbohydrates, caloric expenditure due to exercise, one or more blood glucose values, wherein each of the one or more blood glucose values is associated with a time increment, and wherein the previous blood glucose data has been modified to augment a minority class within the previous blood glucose data.

3. The method of claim 1, wherein the predictive model is associated with one or more ordinary differential equations (ODEs) and wherein one or more coefficients of the one or more ODEs is associated with a parameter of the predictive model.

4. The method of claim 1, wherein the one or more future blood glucose values comprises at least one blood glucose value from 5-60 minutes in the future.

5. The method of claim 1, wherein causing administration of the insulin comprises one or more of causing in insulin pump to inject exogenous insulin or outputting a notification configured to prompt a user to administer insulin.

6. The method of claim 1, wherein the threshold is one or more of below 70 mg/dl or above 120 mg/dl.

7. The method of claim 1, further comprising training the predictive model using previous blood glucose data from the patient further comprises fixing weights and biases in the feature block and tuning weights and biases in a feed-forward neural network (FNN).

8. An apparatus, comprising:
one or more processors; and
memory storing processor executable instructions that, when executed by the one or more processors, cause the apparatus to:
receive, by a predictive model, current blood glucose data from the patient, wherein the predictive model is trained on previous blood glucose data from a population and previous blood glucose data from a patient;
determine, based on the current blood glucose data, one or more future blood glucose values for the patient, wherein the predictive model; and
determine the one or more future blood glucose values satisfies a threshold; and
based on the one or more future blood glucose values satisfying a threshold, cause an administration of insulin.

9. The apparatus of claim 8, wherein the current blood glucose data comprises one or more blood glucose values, wherein each of the one or more blood glucose values is associated with a time increment, and wherein the previous blood glucose data has been modified to augment a minority class within the previous blood glucose data.

10. The apparatus of claim 8, wherein the predictive model is associated with one or more ordinary differential equations (ODEs) and wherein one or more coefficients of the one or more ODEs is associated with a parameter of the predictive model.

11. The apparatus of claim 8, wherein the processor executable instructions that, when executed by the one or more processors, cause the apparatus to cause the administration of insulin, further cause the apparatus to one or more of cause in insulin pump to inject exogenous insulin or cause a user device to output a notification configured to prompt a user to administer insulin.

12. The apparatus of claim 8, wherein the threshold is one or more of below 70 mg/dl or above 120 mg/dl.

13. The apparatus of claim 8, wherein the one or more future blood glucose values comprises at least one blood glucose value from 5-60 minutes in the future.

14. The apparatus of claim 8, wherein the processor executable instructions that, when executed by the one or more processors, further cause the apparatus to train the predictive model using previous blood glucose data from the patient further comprises fixing weights and biases in the feature block and tuning weights and biases in a feed-forward neural network (FNN).

15. A system comprising:
a first computing device configured to:
receive, by a predictive model, current blood glucose data from the patient, wherein the predictive model is trained on previous blood glucose data from a population and previous blood glucose data from a patient;
determine, based on the current blood glucose data, one or more future blood glucose values for the patient, wherein the predictive model; and
determine the one or more future blood glucose values satisfies a threshold;
based on the one or more future blood glucose values satisfying a threshold, cause an administration of insulin; and
an output device configured to:
output, based on the one or more future blood glucose values satisfying the threshold, an alarm.

16. The system of claim 15, wherein the current blood glucose data comprises one or more blood glucose values, wherein each of the one or more blood glucose values is associated with a time increment, and wherein the previous blood glucose data has been modified to augment a minority class within the previous blood glucose data.

17. The system of claim 15, wherein the predictive model is associated with one or more ordinary differential equations (ODEs) and wherein one or more coefficients of the one or more ODEs is associated with a parameter of the predictive model.

18. The system of claim 15, wherein the computing device is further configured cause the apparatus to cause the administration of insulin, further cause the apparatus to one or more of cause in insulin pump to inject exogenous insulin or cause a user device to output a notification configured to prompt a user to administer insulin.

19. The system of claim 15, wherein the threshold is one or more of below 70 mg/dl or above 120 mg/dl.

20. The system of claim 15, wherein the one or more future blood glucose values comprises at least one blood glucose value from 5-60 minutes in the future.
